(19) United States
(12) Patent Application Publication
(10) Pub. No.: US 2023/0321245 A1
(43) Pub. Date: Oct. 12, 2023

(54) PHARMACEUTICAL COMPOSITION

(71) Applicant: MEDINCELL S.A., Jacou (FR)

(72) Inventors: Juliette Serindoux, Jacou (FR); Adolfo Lopez Noriega, Jacou (FR); Feifei Ng, Jacou (FR); Marie-Emérentienne Cagnon, Jacou (FR); Victor Nicoulin, Jacou (FR); Silvio Curia, Jacou (FR); Jean-Manuel Cros, Jacou (FR)

(21) Appl. No.: 18/012,795

(22) PCT Filed: Jul. 2, 2021
(86) PCT No.: PCT/EP2021/068376

§ 371 Date: Dec. 23, 2022

(30) Foreign Application Priority Data
Jul. 6, 2020 (FR) 2010340.4

(51) Int. Cl. A61K 47/34; A61K 31/5415; A61K 31/18; A61K 9/00

(52) U.S. Cl. CPC A61K 47/34; A61K 9/0024; A61K 31/18; A61K 31/5415

(57) ABSTRACT

The present invention provides a pharmaceutical composition comprising:
a multi-branched copolymer comprising at least three polyester arms, wherein the polyester is poly(ε-caprolactone-co-lactic acid), attached to a central core which comprises a polyether, and wherein the multi-branched copolymer is substantially insoluble in aqueous solution, further comprising at least one pharmaceutically active ingredient, and a pharmaceutically acceptable organic solvent in an amount of at least 20% (w/w %) of the total composition.

### FIELD OF THE INVENTION

The present invention relates to controlled release drug delivery or pharmaceutical compositions, in particular pharmaceutical compositions suitable for generating an in situ depot. Specifically, the present invention relates to a pharmaceutical composition comprising a multi-branched copolymer comprising at least three poly(ε-caprolactone-co-lactic acid) arms attached to a central core which comprises a polyether, and wherein the multi-branched copolymer is substantially insoluble in aqueous solution, further comprising at least one pharmaceutically active ingredient, and a pharmaceutically acceptable organic solvent in an amount of at least 20% (w/w %) of the total composition.

### BACKGROUND OF THE INVENTION

WO2012/090070A describes a solvent-exchange in situ forming depot (ISFD) technology comprising a mixture of linear (m)PEG-polyester dissolved in a biocompatible organic solvent. Upon injection, the solvent diffuses and the polymers, insoluble in water, precipitate and form a depot that can entrap an active pharmaceutical ingredient (API). The drug substance is released during a prolonged time from this depot.

There is still a need to provide an improved technology for sustained release. The use of branched PEG-polyester copolymers has been identified as a potential way of improving presently used technologies which suffer from drawbacks such as high viscosity, high injectability values, and relatively slow degradation kinetics.

Star-shaped PEG-polyester copolymers are branched structures consisting of several (three or more) linear chains connected to a central core. Star-shaped copolymers can be classified into two categories: star-shaped homopolymers or star-shaped copolymers. (S. J. Buwalda et al., Influence of amide versus ester linkages on the properties of eight-armed PEG-PLA star block copolymer hydrogels, Biomacromolecules 11 (2010) 224-232). Star-shaped homopolymers consist in a symmetrical structure comprising radiating arms with identical chemical composition and similar molecular weight. Star-shaped copolymers consist of a symmetrical structure comprising radiating arms with similar molecular weight but composed of at least two different monomers.

Star-shaped (also known as multi-arm or multi-branched) copolymers are described in Cameron et al, Chemical Society Reviews, 40, 1761, 2011, and in Burke et al, Biomacromolecules, 18, 728, 2017).

Hiemstra et al, Biomacromolecules, 7, 2790, 2006; Buwalda et al, Biomacromolecules, 11, 224, 2010; Calucci et al, Langmuir, 26, 12890, 2010; Mayadunne et al, US2016/0058698A1, 2016 describe thermogelling aqueous systems.

EP1404294B1 describes the utilization of branched (co)polymers for formulating in situ forming depots (ISFDs) by solvent exchange.

Accordingly, there is a need to provide new solvent-exchange ISFD formulations based on star-shaped copolymers with lower viscosity, improved injectability, improved or different release characteristics of the API or depot degradation kinetics.

### SUMMARY OF THE INVENTION

The present invention provides a pharmaceutical composition comprising:

a multi-branched copolymer comprising at least three polyester arms, wherein the polyester is poly(ε-caprolactone-co-lactic acid), attached to a central core which comprises a polyether, and wherein the multi-branched copolymer is substantially insoluble in aqueous solution, further comprising at least one pharmaceutically active ingredient, and a pharmaceutically acceptable organic solvent in an amount of at least 20% (w/w %) of the total composition.

Typically, the molecular weight of the polyether is 10 kDa or less, 5 kDa or less, 4 kDa or less, 3 kDa or less, or 2 kDa or less, or 1 kDa or less, or 0.5 kDa or less, optionally at least 0.2 kDa.

The present invention also provides a pharmaceutical composition comprising:

a multi-branched copolymer comprising at least three polyester arms, wherein the polyester is poly(ε-caprolactone-co-lactic acid), attached to a central core which comprises a polyether, and wherein the molecular weight of the polyether is 10 kDa or less, 5 kDa or less, preferably 4 kDa or less, 3 kDa or less, or 2 kDa or less, or 1 kDa or less, or 0.5 kDa or less, further comprising at least one pharmaceutically active ingredient, and a pharmaceutically acceptable organic solvent in an amount of at least 20% (w/w %) of the total composition.

The above-mentioned compositions are suitable for forming an in situ depot.

It has been surprisingly found that formulations based on multi-branched or star-shaped copolymers have a lower viscosity and improved injectability, whilst at the same time providing improved or different release profiles of the pharmaceutically active ingredient, compared to formulations comprising linear copolymer analogues alone.

Typically, the multi-branched copolymer is substantially insoluble in aqueous solution, optionally in water.

In a preferred embodiment, the multi-branched copolymer has less than 15 mg/mL, optionally less than 10 mg/mL, less than 5 mg/mL, less than 2 mg/mL, or less than 1 mg/mL solubility in aqueous solution, optionally in water. Typically, the solubility is measured at 37° C.

Typically, the multi-branched copolymer is of formula A(B)ₙ wherein A represents the central core and B represents the polyester arms and n is an integer of at least 3. In embodiments of the invention n is at least 4, or at least 6, or at least 8. n may be 3, 4, 6 or 8. Preferably, n is 3 or 4.

In one embodiment the central core is a multi-branched polyether which is derivable from poly(ethylene glycol) (PEG) and a polyol. Typically, the polyol comprises at least three hydroxyl groups. The polyol is typically a hydrocarbon functionalized with at least three hydroxyl groups, optionally 3, 4, 5, 6 or 8 hydroxyl groups. In some embodiments the polyol further comprises one or more ether groups. Preferably the polyol is pentaerythritol (PE), dipentaerythritol (DPE), trimethylolpropane (TMP), glycerol, hexaglycerol, erythritol, xylitol, di(trimethylolpropane) (diTMP), sorbitol, or inositol.

In preferred embodiments the multi-branched polyether has any of Formulae 1 to 4:

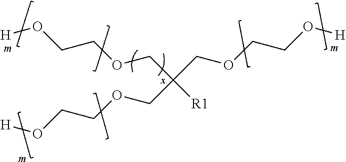

wherein R₁ is

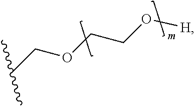

H or alkyl, x is 0 or 1 and m is an integer between 2 and 76

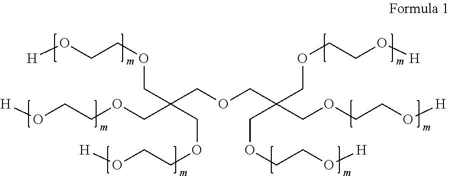

wherein m is an integer between 5 and 40

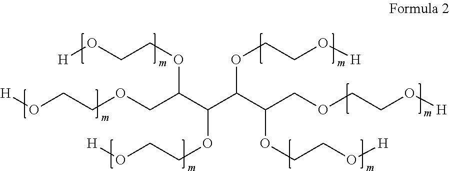

wherein m is an integer between 5 and 40

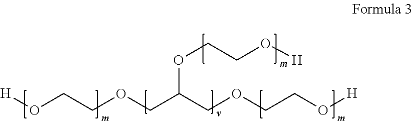

wherein m is an integer between 25 and 30 and v is 6

In one embodiment the multi-branched polyether has Formula 1, x is 1 and R₁ is alkyl, optionally ethyl.

In one embodiment the multi-branched polyether has Formula 1, x is 1 and R₁ is

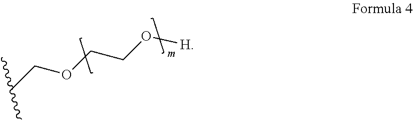

In one embodiment the multi-branched polyether has Formula 1, x is 0 and R₁ is H.

The polyester arms are typically formed by reacting a precursor or monomer of the polyester with the polyether core. For example, the polyether is reacted with D,L-lactide and ε-caprolactone.

In preferred embodiments each branch of the multi-branched polyether has a terminal reactive group capable of reacting with a polyester or monomer or precursor thereof. Typically, the terminal reactive group is a hydroxyl group or an amine group, but preferably a hydroxyl group.

In one embodiment the multi-branched copolymer is obtainable by reacting a multi-branched polyether as defined above with D,L-lactide and ε-caprolactone. The multi-branched copolymer may be obtainable by ring-opening polymerization of the D,L-lactide and ε-caprolactone initiated by the multi-branched polyether.

In one embodiment the number of ester repeat units in each arm is independently in the range of 5 to 230, optionally 10 to 115, optionally 10 to 90, and wherein the ratio of lactic acid repeat units to hexanoate repeat units is in the range of 25/75 to 99/1.

In one embodiment the multi-branched copolymer has Formula 5 or Formula 6 or Formula 7 or Formula 8:

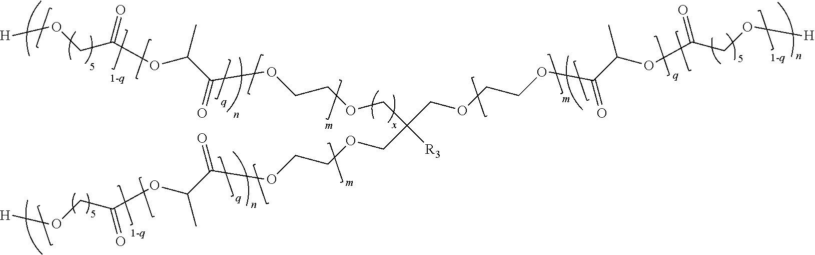

wherein R₃ is

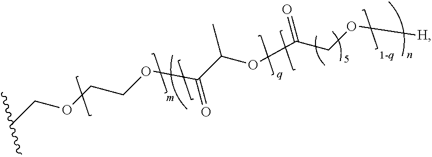

H or alkyl, x is 0 or 1, m is an integer between 2 and 76; n is an integer between 5 and 230 and q is between 0.25 and 0.99

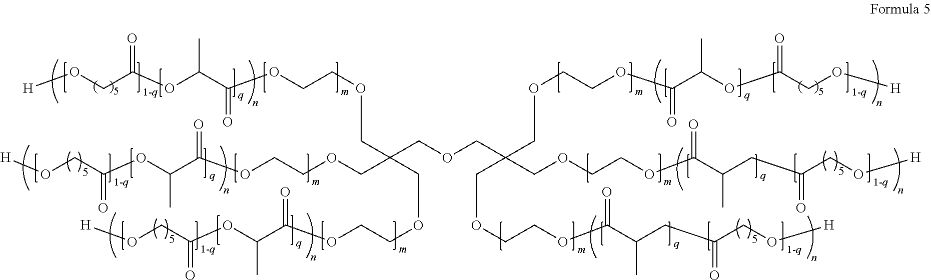

Wherein m is an integer between 5 and 40, n is an integer between 10 and 115 and q is between 0.25 and 0.99

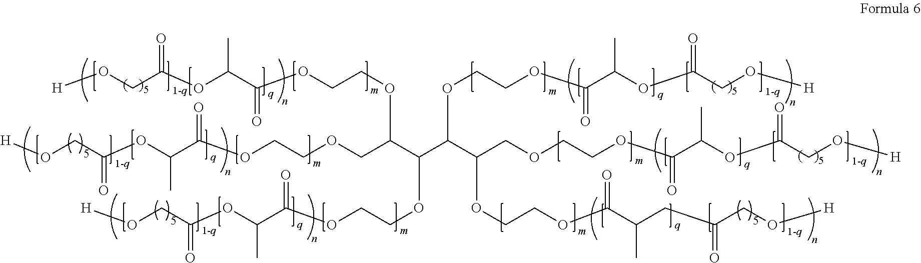

Wherein m is an integer between 5 and 40, n is an integer between 10 and 115 and q is between 0.25 and 0.99

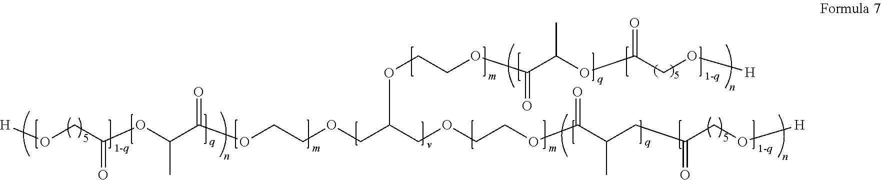

Wherein m is an integer between 25 and 30; n is an integer between 10 and 90; q is between 0.25 and 0.99 and v is 6

In one embodiment, the multi-branched copolymer has Formula 5, x is 1 and R₃ is alkyl, optionally ethyl.

In one embodiment the multi-branched copolymer has Formula 5, x is 1 and R₃ is

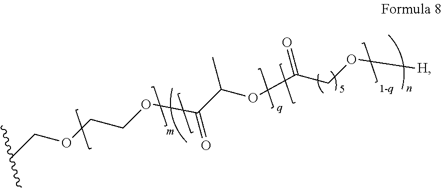

wherein m is an integer between 2 and 76; n is an integer between 5 and 230 and q is between 0.25 and 0.99.

In one embodiment, the multi-branched copolymer has Formula 5, x is 0 and R₃ is H.

In one embodiment the multi-branched copolymer has Formula 5, the polyether core has a molecular weight of 2 kDa and the ester repeat unit to ethylene oxide molar ratio is 4 or 6.

In one embodiment the multi-branched copolymer has Formula 5, the polyether core has a molecular weight of 0.45 kDa and the ester repeat unit to ethylene oxide molar ratio is 6.

In a preferred embodiment, the molecular weight of the polyether ranges from 0.5 kDa to 10 kDa, optionally 1 kDa to 10 kDa, preferably 2 kDa to 10 kDa, preferably 2 kDa to 5 kDa, or most preferably 0.5 kDa to 2 kDa.

In preferred embodiments the ester repeat unit to ethylene oxide molar ratio of the multi-branched copolymer in the composition is from 1 to 10, preferably from 2 to 6.

The composition of the invention comprises a pharmaceutically acceptable organic solvent in an amount of at least 20% (w/w %) of the total composition. Typically, the organic solvent is a biocompatible organic solvent, optionally wherein the amount of said vehicle is at least 25%, or at least 35% (w/w %) of the total composition. Preferably, the pharmaceutically acceptable vehicle is selected from the group of: benzyl alcohol, benzyl benzoate, dimethyl isosorbide (DMI), dimethyl sulfoxide (DMSO), ethyl acetate, ethyl benzoate, ethyl lactate, glycerol formal, methyl ethyl ketone, methyl isobutyl ketone, N-ethyl-2-pyrrolidone, N-methyl-2-pyrrolidinone (NMP), pyrrolidone-2, tetraglycol, triacetin, tributyrin, tripropionin, glycofurol and mixtures thereof.

In one embodiment the pharmaceutically active ingredient is hydrophobic. By this is meant a pharmaceutically active ingredient having positive log P or log D values and aqueous solubility at physiological pH (pH 7.0 to 7.4) below 1 mg/mL.

In a preferred embodiment the pharmaceutically active ingredient is meloxicam, tamsulosin, or combinations thereof.

In one embodiment the at least one pharmaceutically active ingredient is present in an amount of from 0.05% to 60%, optionally 0.05% to 40%, optionally 5% to 30%, optionally 5% to 25%, optionally 5% to 20%, optionally 10% to 20% (w/w %) of the total composition.

In a preferred embodiment, the at least one pharmaceutically active ingredient is in the form of suspension at a temperature between 10 and 37° C.

In a preferred embodiment the composition is an injectable liquid.

In a preferred embodiment, the multi-branched copolymer is present in an amount of 20% to 70%, optionally 20% to 60%, optionally 30% to 60%, optionally 30% to 50% (w/w %) of the total composition

In one embodiment the compositions are as defined in Table 2.

Typically, the release of at least one pharmaceutically active ingredient can be modulated by the composition.

In one embodiment the composition is suitable to deliver a pharmaceutically active ingredient to a subject for at least 1 day, optionally at least 3 days, optionally at least 7 days, optionally at least 30 days, optionally at least 90 days, optionally at least 180 days, optionally at least 1 year.

In an additional aspect, the present invention provides a method of producing a pharmaceutical composition as defined above, said method comprising dissolving a multi-branched copolymer as defined above in a pharmaceutically acceptable vehicle, such as a solvent, and subsequently adding a pharmaceutically active ingredient to the composition.

In a further aspect, the invention provides a bioresorbable depot which is produced ex vivo or in situ by contacting the composition as defined above with an aqueous medium, water or body fluid. The depot is bioresorbable in the sense that the PCLA moieties degrade in vivo, and that the PEG is assimilated by the body and excreted.

In a final aspect, provided is a method for the controlled release of a pharmaceutically active ingredient comprising administering the composition as defined above and allowing a solvent-exchange in situ depot to be formed in vivo.

### DETAILED DESCRIPTION

As used herein the term “bioresorbable” or “biodegradeable” means that the block copolymers undergo hydrolysis in vivo to form their constituent (m)PEG and oligomers or monomers or repeat units derived from the polyester block. For example, PCLA undergoes hydrolysis to form 6-hydroxycaproic acid (6-hydroxyhexanoic acid) and lactic acid. The result of the hydrolysis process leads to a progressive mass loss of the depot and ultimately to its disappearance.

The term “multi-branched copolymer” means a polymer with at least three polyester arms attached to a central core which comprises a polyether. The polyester arms may be referred to as “branches”, “arms” or “chains”. The term “multi-branched copolymer” has the same meaning as the term “star copolymer” or “star-shaped copolymer” or “multi-arm copolymer” and these terms are used interchangeably throughout.

Typically, the molecular weight of the polyether is 10 kDa or less, 5 kDa or less, 4 kDa or less, 3 kDa or less, or 2 kDa or less, or 1 kDa or less or 0.5 kDa or less. Preferably, the polyether has a molecular weight of at least 0.2 kDa. The molecular weight is the number average molecular weight (Mn) as measured by Gel Permeation Chromatography (GPC) or Matrix Assisted Laser Desorption Ionization-Time of Flight Mass Spectrometry (MALDI-TOF MS). GPC using a calibration curve obtained from polystyrene standards is the preferred method of measuring Mn.

Typically, the multi-branched copolymer is of formula A(B)ₙ wherein A represents the central core and B represents the polyester arms and n is an integer of at least 3. In embodiments of the invention n is at least 4, or at least 6, or at least 8. Preferably, n is 4. An example of the structure of a multi-branched PEG-PCLA block copolymer with n=3 or n=4 is provided below.

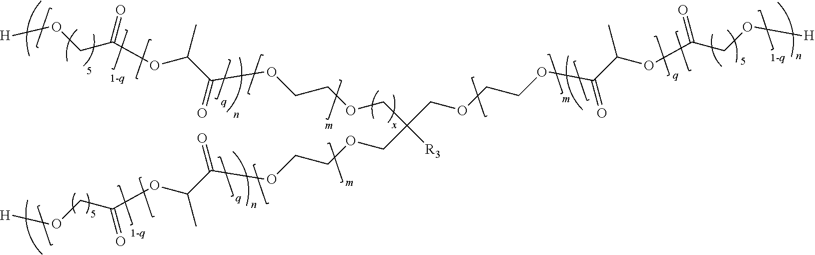

wherein R₃ is

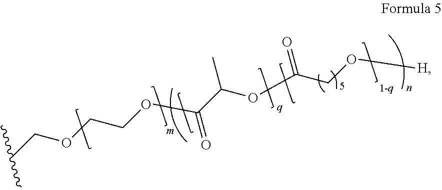

H or alkyl, x is 0 or 1, m is an integer between 2 and 76; n is an integer between 5 and 230 and q is between 0.25 and 0.99;

A polyol is an organic compound comprising a plurality of hydroxyl groups. Typically, the polyol has at least three hydroxyl groups. Typically, the polyol is a hydrocarbon functionalized with at least three hydroxyl groups, for example 3, 4, 5, 6, or 8 hydroxyl groups. The polyol may also comprise one or more ether groups. Typically, the polyol is pentaerythritol (PE), dipentaerythritol (DPE), trimethylolpropane (TMP), glycerol, hexaglycerol, erythritol, xylitol, di(trimethylolpropane) (diTMP), sorbitol, or inositol.

A polyether is an organic compound comprising a plurality of ether groups.

In a preferred embodiment the central core is a multi-branched polyether which is derivable (obtainable) from poly(ethylene glycol) (PEG) and a polyol. For example, the multi-branched polyether may be formed by reaction of ethylene oxide with a polyol. The multi-branched polyether is obtainable by reaction of ethylene oxide with a polyol. The multi-branched polyether may be referred to as a star-shaped PEG. The ethylene oxide reacts with a hydroxyl group of a polyol to form a PEG arm. For example, pentaerythritol may be reacted with ethylene oxide to form the four arm or four branched polyether set out below in Formula 1, wherein x is 1 and R₁ is

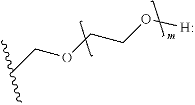

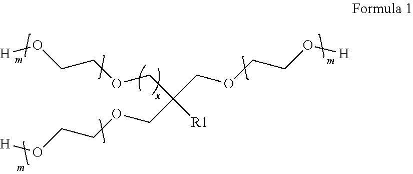

and m is an integer between 2 and 76

3-arm polyethers wherein x is 1 and R₁ is H or alkyl are formed by reaction of ethylene oxide with trimethylolmethane or trimethylolpropane, respectively.

3-arm polyethers wherein x is 0 and R₁ is H are formed by reaction of ethylene oxide with glycerol.

In an alternative embodiment the multi-branched polyether is a 6-arm, or 6-branched polyether as set out below in Formula 2 and Formula 3:

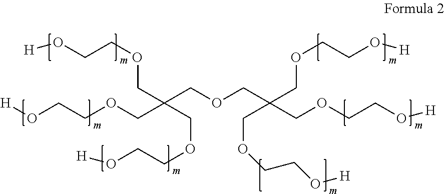

wherein m is an integer between 5 and 40

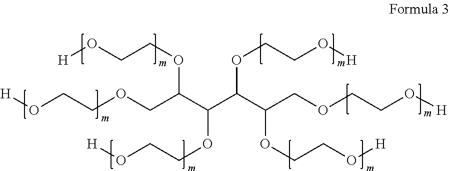

wherein m is an integer between 5 and 40

In an alternative embodiment the multi-branched polyether is an eight-arm or eight branched polyether (v=6) as set out below in Formula 4:

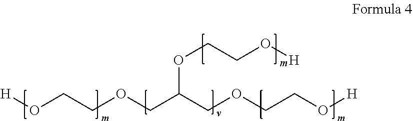

wherein m is an integer between 25 and 30 and v is 6

Typically, each branch of the multi-branched polyether has a terminal hydroxyl group, however other terminal reactive groups capable of reacting with a polyester or monomers or precursors thereof may also be contemplated. The polyester is poly(ε-caprolactone-co-lactic acid) (PCLA). Typically, the terminal hydroxyl group of each branch of the multi-branched polyether reacts with a monomer or precursor of the polyester to form a polyester arm. For example, D-L-lactide and ε-caprolactone may react with the multi-branched polyether to form a PCLA arm.

In one embodiment the number of ester repeat units in each arm is independently in the range of 5 to 230, optionally 10 to 115, optionally 10 to 90 and wherein the ratio of lactic acid repeat units to hexanoate repeat units is in the range of 25/75 to 99/1. When the polymer has 3 or 4 ester arms, preferably the number of ester repeat units in each arm is independently in the range of 5 to 230. When the polymer has 6 polyester arms, preferably the number of ester repeat units in each arm is independently in the range of 10-115. When the polymer has 8 polyester arms, preferably the number of ester repeat units in each arm is independently in the range of 10-90.

In a preferred embodiment, the molecular weight of the polyether ranges from 0.5 kDa to 10 kDa, optionally 1 kDa to 10 kDa, preferably 2 kDa to 10 kDa, preferably 2 kDa to 5 kDa or most preferably 0.5 kDa to 2 kDa.

The term “depot injection” means an injection of a flowing pharmaceutical composition, usually subcutaneous, intradermal or intramuscular that deposits a drug in a localized mass, such as a solid mass, called a “depot”. The depots as defined herein are in situ forming upon injection. Thus, the formulations can be prepared as solutions or suspensions and can be injected into the body.

An “in situ depot” is a solid, localized mass formed by precipitation of the pharmaceutical composition after injection of the composition into the subject. The pharmaceutical composition comprises a multi-branched copolymer which is substantially insoluble in aqueous solution. Thus, when the pharmaceutical composition comes in contact with the aqueous environment of the human or animal body, a phase inversion occurs causing the composition to change from a liquid to a solid, i.e. precipitation of the composition occurs, leading to formation of an “in situ depot”.

An “in situ depot” can be clearly distinguished from hydrogel pharmaceutical formulations described in the prior art.

Hydrogels can be formed from star polymers comprising a polyether core and PCLA branches. Certain star polymers comprising a polyether core and PCLA branches can form micelles in aqueous solution. The hydrophobic PCLA outer blocks associate with neighboring micelles to form a network of linked micelles or large aggregates, giving rise to gels under specific temperature and concentration ranges. Hydrogels have three-dimensional networks that are able to absorb large quantities of water. The polymers making up hydrogels are soluble in aqueous solution. By contrast, the multi-branched polymers used in the present invention are substantially insoluble in aqueous solution. The pharmaceutical compositions of the invention are free of water, or substantially free of water. For example, the pharmaceutical compositions of the invention comprise less than 0.5% w/w water.

Typically, the multi-branched copolymer is substantially insoluble in aqueous solution. Typically, this means that the multi-branched copolymer has less than 15 mg/mL, optionally less than 10 mg/mL, less than 5 mg/mL, less than 2 mg/mL, optionally less than 1 mg/mL solubility in aqueous solution, optionally in water. Typically, the solubility is measured at 37° C.

In a preferred embodiment, the solubility of the multi-branched copolymer in water was determined as follows:

500 mg of copolymer were put in an empty 20 mL vial. 5 mL of ultra-pure water were added, the vial was put at 37° C. under continuous vortexing for 2 hours. Then, the vial was centrifuged 10 min at 3000 rpm. The supernatant was transferred to another vial of known weight which was placed at −80° C. overnight, prior to lyophilization for 24 h. The amount of solubilized copolymer was determined as the difference of weight of the empty vial and the lyophilized one.

Temperature sensitive hydrogels, or thermogels as described in the prior art are typically solid at a specific narrow temperature range, for example 30 to 35° C., and this solidification is reversible. By contrast, the in situ depot formed in the present invention is solid when injected in an aqueous medium over a much broader temperature range, for example 20 to 37° C.

In addition, hydrogels based on PEG and PCLA typically enable release of an API for a shorter period of time than depots formed by the composition of the present invention.

The compositions of the invention comprise a pharmaceutically acceptable organic solvent in an amount of at least 20% (w/w %) of the total composition. The solvent is typically a biocompatible solvent. Preferably, the pharmaceutically acceptable vehicle is selected from the group of: benzyl alcohol, benzyl benzoate, dimethyl isosorbide (DMI), dimethyl sulfoxide (DMSO), ethyl acetate, ethyl benzoate, ethyl lactate, glycerol formal, methyl ethyl ketone, methyl isobutyl ketone, N-ethyl-2-pyrrolidone, N-methyl-2-pyrrolidinone (NMP), pyrrolidone-2, tetraglycol, triacetin, tributyrin, tripropionin, glycofurol, and mixtures thereof. The amount of said solvent is typically at least 25%, or at least 35% (w/w %) of the total composition. The amount of solvent may be 20% to 60%, optionally 30% to 60% preferably 40% to 60% (w/w %) of the total composition. The amount of solvent provides the balance up to 100 w/w %, taking note of the presence of the multi-branched copolymer, the pharmaceutically active ingredient and any other excipients.

The composition comprises at least one pharmaceutically active ingredient.

In one embodiment the pharmaceutically active ingredient is hydrophobic.

In a preferred embodiment the pharmaceutically active ingredient is meloxicam, tamsulosin or combinations thereof.

In one embodiment the at least one pharmaceutically active ingredient is present in an amount of from 0.05% to 60%, optionally 0.05% to 40%, optionally 5% to 30%, optionally 5% to 25%, optionally 5% to 20%, optionally 10% to 20% (w/w %) of the total composition.

The compositions of the invention are particularly suitable for formulating suspensions of pharmaceutically active ingredients. A suspension is a heterogeneous mixture in which the solute particles (for example API) do not dissolve or completely dissolve in a solvent but get suspended throughout the bulk of the solvent. Suspensions may form when the API has low solubility in a solvent or when the API is formulated at high concentration. The soluble fraction is defined as the percentage of solubilized API over the total amount of API. This quantity can be measured using an appropriate UPLC method.

In a preferred embodiment, the at least one pharmaceutically active ingredient is in the form of suspension at a temperature between 10 and 37° C.

It has been demonstrated that star-shaped PEG-PCLA copolymers have a lower viscosity and injectability relative to linear block copolymers comprising PEG and PCLA or relative to star or linear block copolymers comprising poly(lactic acid) (PLA) or poly(lactic acid-co-glycolic acid) (PLGA) as disclosed in WO2012/090070A or PCT/EP2020/050333. This feature makes star-shaped PEG-PCLA copolymer compositions of the invention particularly suitable for the use with at least one pharmaceutically active ingredient in the form of suspension.

In a preferred embodiment the composition is an injectable liquid.

The multi-branched copolymer is preferably present in an amount of 20% to 70%, optionally 20% to 60%, optionally 30% to 60% (w/w %), optionally 30% to 50% (w/w %) of the total composition.

Typically, the ester repeat unit to ethylene oxide molar ratio in the composition is from 1 to 10, preferably from 2 to 6.

Typically, the release of at least one pharmaceutically active ingredient can be modulated by the composition.

In one embodiment the composition is suitable to deliver a pharmaceutically active ingredient to a subject for at least 1 day, optionally at least 3 days, optionally at least 7 days, optionally at least 30 days, optionally at least 90 days, optionally at least 180 days, optionally at least 1 year.

In a further aspect, the present invention provides use of the pharmaceutical composition as defined above to modulate the kinetics of release of at least one pharmaceutically active ingredient.

In an additional aspect, the present invention provides a method of producing a pharmaceutical composition as defined above, said method comprising dissolving a multi-branched copolymer as defined above in a pharmaceutically acceptable vehicle, and subsequently adding a pharmaceutically active ingredient to the composition.

In a further aspect, the invention provides a bioresorbable depot which is produced ex vivo or in situ by contacting the composition as defined above with an aqueous medium, water or body fluid.

In a final aspect, provided is a method for the controlled release of a pharmaceutically active ingredient comprising administering the composition as defined above to a subject and allowing an in situ depot to be formed in vivo.

The pharmaceutical composition is preferably suitable for parenteral administration. The term “parenteral administration” encompasses intramuscular, intraperitoneal, intra-abdominal, subcutaneous, intravenous and intraarterial. It also encompasses intradermal, intracavernous, intravitreal, intracerebral, intrathecal, epidural, intra-articular, and intraosseous administration.

The subject may be an animal or a plant. The term “animals” encompasses all members of the Kingdom Animalia. The animal may be a human or non-human animal.

As used herein the term “plant” encompasses all members of the Plant Kingdom.

“Pharmaceutically active ingredient” means a drug or medicine for treating or preventing various medical illnesses. For the purposes of the present application the term “active principle” has the same meaning as “active ingredient”. Thus, the terms active ingredient, active principle, drug or medicine are used interchangeably. The term Active Pharmaceutical Ingredient, or “API” is also used. The term drug or active ingredient as used herein includes without limitation physiologically or pharmacologically active substances that act locally or systemically in the body of an animal or plant.

As used herein “disease” means any disorder in a human, animal or plant caused by infection, diet, or by faulty functioning of a process.

The term “spatial formulation” encompasses any formulation that can be applied on or into the animal or plant body and do not necessarily have to be administered through a syringe.

As used herein “repeat units” are the fundamental recurring units of a polymer.

As used herein “polyethylene glycol”, as abbreviated PEG throughout the application, is sometimes referred to as poly(ethylene oxide) or poly(oxyethylene) and the terms are used interchangeably in the present invention.

The abbreviation “PCLA” refers to poly(ε-caprolactone-co-lactic acid).

The abbreviation “PEG” refers to poly(ethylene glycol).

The abbreviation “CL” refers to ε-caprolactone or hexanoate repeat units. Caprolactone refers to the closed ring used as a reactant for the polyester synthesis. Once opened it reacts and leads to the formation of hexanoate repeat units

The copolymers have been named as follows:

DLkC-szPxRp stands for a star-shaped PEG-PCLA copolymer composed of D,L-lactide (DL) and ε-caprolactone (C) where k is the molar ratio of LA compare to CL, z represents the number of arms, x is the number average molecular weight of the polyether core formed from the reaction of a polyol and PEG (often referred to as the “star-shaped PEG”) in kDa and p is the [(lactic acid+hexanoate)/ethylene oxide] [(LA+CL)/EO] molar ratio and allows the calculation of the PCLA chain length within the copolymer.

As an example, DL80C-s4P2R6 is a 4-arm star-shaped PEG-PCLA copolymer with a 2 kDa star-shaped PEG block with an overall [(LA+CL)/EO] molar ratio of 6. Each polyester arm is composed of 80% LA.

DLkC-PxRp stands for a linear PCLA-PEG-PCLA triblock polymer composed of D,L-lactide (DL) and ε-caprolactone (C) and where k, x and p provide the same information as in PEG-PCLA star-shaped copolymers, namely k represents the molar ratio of LA compare to CL, x is the molecular weight of the PEG chain in kDa and p is the [(LA+CL)/EO] molar ratio.

DLkC-dPxRp stands for a linear mPEG-PCLA diblock polymer composed of D,L-lactide (DL) and ε-caprolactone (C) and where k, x and p provide the same information as in PEG-PCLA star-shaped copolymers, namely k represents the molar ratio of LA compare to CL, x is the molecular weight of the PEG chain in kDa and p is the [(LA+CL)/EO] molar ratio.

The “injectability” of a formulation, as used herein, is defined by the force needed in Newtons (N) to inject a formulation using pre-determined parameters. These parameters include injection speed, injection volume, injection duration, syringe type or needle type and the like. These parameters may vary based on at least one pharmaceutically active ingredient used, or the desired method of administration such as subcutaneous, intra-ocular, intra-articular and the like. They may be adjusted based on the at least one pharmaceutically active ingredient present within the formulations, to be able to observe the differences and fluctuations between the formulations. The injectability must be kept low such that the formulation can be easily administered by a qualified healthcare professional in an acceptable timeframe. An acceptable injectability value may be from 0.1 N to 20 N with the measurement method described below, with an injectability of from 0.1 N to 10 N being most preferred. A non-optimal injectability may be greater than 20 N to 30 N. Formulations are hardly injectable from 30 to 40 N and non-injectable above 40 N. Injectability may be measured using a texturometer, preferably a Lloyd Instruments FT plus texturometer, using the following analytical conditions: 500 μL of formulation are injected through a 1 mL syringe, a 23 G 1″ Terumo needle with a 1 mL/min flow rate as described in example 4.

“Viscosity,” by definition and as used herein, is a measure of a resistance of a fluid to flow and gradual deformation by shear stress or tensile strength. It describes the internal friction of a moving fluid. For liquids, it corresponds to the informal concept of “thickness”. By ‘dynamic viscosity” is meant a measure of the resistance to flow of a fluid under an applied f79567orce. The dynamic velocity can range from 1 mPa·s. to 3000 mPa·s or 5 mPa·s to 2500 mPa·s or 10 mPa·s to 2000 mPa·s or 20 mPa·s to 1000 mPa·s. Dynamic viscosity is determined using an Anton Paar Rheometer equipped with cone plate measuring system. Typically, 700 μL of studied formulation are placed on the measuring plate. The temperature is controlled at +25° C. The measuring system used is cone plate with a diameter of 50 mm and cone angle of 1 degree (CP50). CP50 is better appropriate to obtain precise values for formulations showing low viscosity. The working range is from 10 to 1000 s⁻¹. After being vortexed for 10 s, formulations are placed at the center of the thermo-regulated measuring plate using a spatula. The measuring system is lowered down and a 0.104 mm gap is left between the measuring system and the measuring plate. 21 viscosity measurement points are determined across the 10 to 1000 s⁻¹ shear rate range. For suspensions, 60 s rest time was set at the measuring position prior the analysis followed by a pre-shearing at 1000 s⁻¹ for 30 s⁻¹. Given values are the ones obtained at 100 s⁻¹.

Representative drugs and biologically active agents to be used in the invention include, without limitation, peptides, proteins, antibodies, fragments of antibodies, desensitizing agents, antigens, vaccines, vaccine antigens, anti-infectives, antidepressants, stimulants, opiates, antipsychotics, atypical antipsychotics, glaucoma medications, antianxiety drugs, antiarrhythmics, antibacterials, anticoagulents, anticonvulsants, antidepressants, antimetics, antifungals, antineoplastics, antivirals, antibiotics, antimicrobials, antiallergenics, anti-diabetics, steroidal anti-inflammatory agents, decongestants, miotics, anticholinergics, sympathomimetics, sedatives, hypnotics, psychic energizers, tranquilizers, hormones, androgenic steroids, estrogens, progestational agents, humoral agents, prostaglandins, analgesics, corticosteroids, antispasmodics, antimalarials, antihistamines, cardioactive agents, non-steroidal anti-inflammatory agents, antiparkinsonian agents, antihypertensive agents, beta-adrenergic blocking agents, nutritional agents, gonadotrophin releasing hormone agonists, insecticides, anti-helminthic agents or combinations thereof.

The pharmaceutically active ingredient may be meloxicam, tamsulosin, or combinations thereof.

Combinations of drugs can be used in the biodegradable drug delivery composition of this invention. For instance, if one needs to treat Lupus erythematosus, non-steroidal anti-inflammatory agents and corticosteroids can be administered together in the present invention.

Veterinary medicaments such as medicines for the treatment of worms or vaccines for animals are also part of the present invention.

Viral medicaments for plants such as those viruses from Potyviridae, Geminiviridae, the Tospovirus genus of Bunyaviridiae and Banana streak virus are also encompassed by the present invention. Also, medicaments for tobacco mosaic virus, turnip crinkle, barley yellow dwarf, ring spot watermelon and cucumber mosaic virus can be used in the biodegradable drug delivery composition of the invention.

To those skilled in the art, other drugs or biologically active agents that can be released in an aqueous environment can be utilized in the described delivery system. Also, various forms of the drugs or biologically active agents may be used. These include without limitation forms such as uncharged molecules, molecular complexes, salts, ethers, esters, amides, etc., which are biologically activated when injected into the animal or plant or used as a spatial formulation such that it can be applied on or inside the body of an animal or plant or as a rod implant.

The pharmaceutically effective amount of a pharmaceutically active ingredient may vary depending on the pharmaceutically active ingredient, the extent medical condition of the animal or plants and the time required to deliver the pharmaceutically active ingredient. There is no critical upper limit on the amount of pharmaceutically active ingredient incorporated into the polymer solution as long as the solution or suspension has a viscosity which is acceptable for injection through a syringe needle and that it can effectively treat the medical condition without subjecting the animal or plant to an overdose. The lower limit of the pharmaceutically active ingredient incorporated into the delivery system is dependent simply upon the activity of the pharmaceutically active ingredient and the length of time needed for treatment.

In the biodegradable drug delivery composition of the present invention, the pharmaceutically effective amount can be released gradually over an extended period of time. This slow release may be continuous or discontinuous, linear or non-linear and can vary due to the composition of the multi-branched copolymer.

The pharmaceutically active ingredient can be released for a duration of between 1 day to 1 year or longer depending upon the type of treatment needed and the biodegradable drug delivery composition used. In one embodiment the biodegradable drug delivery composition can deliver the pharmaceutically active ingredient for at least 1 day, optionally at least 3 days, optionally at least 7 days. In another embodiment the biodegradable drug delivery composition can deliver the pharmaceutically active ingredient for at least 30 days. In one embodiment the biodegradable drug delivery composition can deliver the pharmaceutically active ingredient for at least 90 days. In yet another embodiment the biodegradable drug delivery composition can deliver a pharmaceutically active ingredient for 1 year or longer.

The biodegradable drug delivery composition can be an injectable liquid, preferably at room temperature, and can be injected through a syringe without excessive force. These biodegradable drug delivery compositions are also in situ forming and biodegradable and turn into solid depots when injected into the animal or plant.

The composition can further comprise a pharmaceutically acceptable carrier, adjuvant or vehicle.

The compositions of the invention comprise an organic solvent in an amount of at least 20% (w/w %) of the total composition. The organic solvent may be selected from the group of: benzyl alcohol, benzyl benzoate, dimethyl isosorbide (DMI), dimethyl sulfoxide (DMSO), ethyl acetate, ethyl benzoate, ethyl lactate, glycerol formal, methyl ethyl ketone, methyl isobutyl ketone, N-ethyl-2-pyrrolidone, N-methyl-2-pyrrolidinone(NMP), 2-pyrrolidone, tetraglycol, triacetin, tributyrin, tripropionin, glycofurol, and mixtures thereof. In one embodiment DMSO, NMP, tripropionin or mixtures thereof can be used as solvents.

### List of Abbreviations

API Active pharmaceutical ingredient
CL ε-caprolactone or hexanoate
Ð Dispersity
DMI Dimethyl isosorbide
DMSO Dimethyl sulfoxide
DPE Dipentaerythritol
DSC Differential Scanning calorimetry
diTMP Di(trimethylolpropane)
EO Ethylene oxide
GA Glycolic acid
GPC Gel permeation chromatography
GPC-TDA Gel permeation chromatography-Triple Detector Array
HPLC High-performance liquid chromatography
ISFD In situ forming depot
IVR In vitro release
KRT Krebs-Ringer-Tris buffer
LA Lactic acid
MALDI-TOF Matrix Assisted Laser Desorption Ionization-Time of Flight
mPEG methoxy-poly(ethylene glycol)
mPEG-PCLA methoxy-poly(ethylene glycol)-b-poly(ε-caprolactone-co-lactic acid)
NA Not applicable
NMP N-methyl-2-pyrrolidinone
PBS Phosphate buffer saline
PCL Poly((ε-caprolactone)
PCLA Poly(ε-caprolactone-co-lactic acid)
PCLA-PEG-PCLA Poly(ε-caprolactone-co-lactic acid)-b-poly(ethylene glycol)-poly(ε-caprolactone-co-lactic acid)
PE Pentaerythritol
PEA Poly(ethylene adipate)
PEG Poly(ethylene glycol)
PGA Poly(glycolic acid)
PHA Poly(hydroxyalkanoate)
PLA Poly(lactic acid)
PLGA Poly(lactic acid-co-glycolic acid)
RT Room Temperature
SD Standard deviation
THF Tetrahydrofuran
TMP Trimethylolpropane
UPLC Ultra-performance liquid chromatography
¹H-NMR Proton nuclear magnetic resonance

### BRIEF DESCRIPTION OF THE DRAWINGS

FIG. 1 shows the percentage in vitro cumulative release of meloxicam over time from three different formulations: Formulation F71 (◯) containing 40.00% of DL90C-s4P2R4 star-shaped copolymer with 2.00% of active pharmaceutical ingredient (API) and 58.00% of DMSO; formulation F75 (□) containing 40.00% of DL90C-s4P2R4 star-shaped copolymer with 10.00% of active pharmaceutical ingredient (API) and 50.00% of DMSO and formulation F62 (Δ) containing 40.00% of DL90C-s4P2R4 star-shaped copolymer with 20.00% active pharmaceutical ingredient (API) and 40.00% of DMSO. In vitro release tests have been conducted according to set up 1 for F71 and set up 2 for F75 and F63 in table 3, example 3. The specific block copolymer formulations are set forth in Table 2 below.

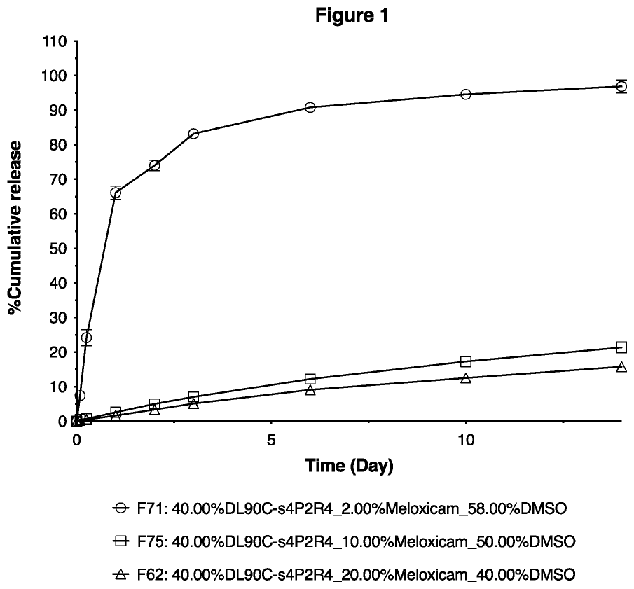

Results demonstrate that the star-shaped copolymer-based formulations allow sustained release with three different API loadings. In particular, data show that API release from formulations in the form of suspensions (F75 and F62) is substantially slower than from a formulation in the form of a solution (F71).

FIG. 2 is a graph showing the percentage in vitro cumulative release of meloxicam over time from F64. Formulation F64 (□) contains 40.00% of DL30C-s4P2R4 star-shaped copolymer with 20.00% of active pharmaceutical ingredient (API) and 40.00% of DMSO. In vitro release tests have been conducted according to set up 2 in table 3, example 3. The specific block copolymer formulations are set forth in Table 2 below.

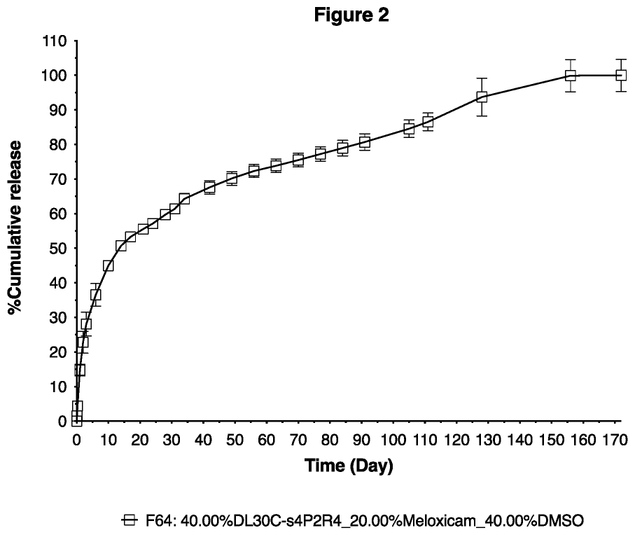

Results demonstrate that star-shaped copolymer-based formulation in accordance with the invention leads to a sustained release of the drug for up to at least 172 days.

FIG. 3 shows the percentage in vitro cumulative release of meloxicam over time from two different formulations: Formulation F79 (□) containing 40.00% of DL80C-P2R4 triblock copolymer with 20.00% of active pharmaceutical ingredient (API) and 40.00% of DMSO and formulation F63 (◯) containing 40.00% of DL80C-s4P2R4 star-shaped copolymer with 20.00% of active pharmaceutical ingredient (API) and 40.00% of DMSO. In vitro release tests have been conducted according to set up 2 in table 3, example 3. The specific block copolymer formulations are set forth in Table 2.

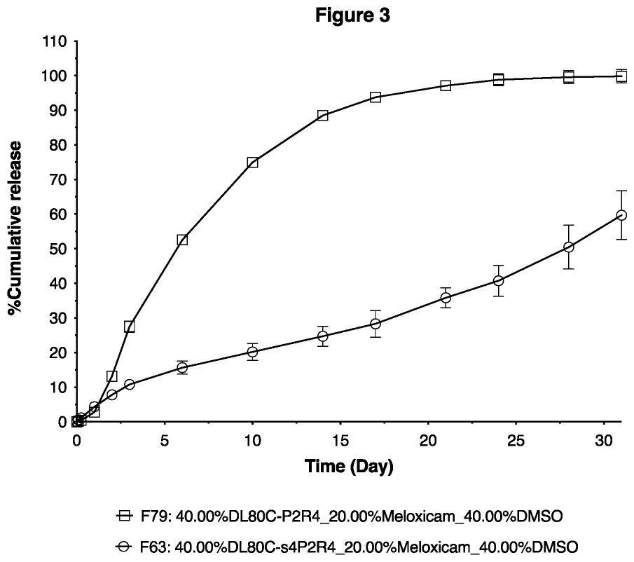

Results indicate that the star-shaped copolymer-based formulation exhibits slower release kinetics compared to the linear copolymer-based formulation with a comparable molecular weight and an identical total copolymer content. Indeed, formulation F63 shows slower release kinetics than formulation F79.

FIG. 4 displays injectability values of formulations F63 and F79. Data demonstrate that for identical loading of copolymer and a comparable molecular weight, the star-shaped copolymer-based formulation has lower injectability than the linear copolymer-based formulation. Indeed, injectability values for formulation F63 are below those of F79. Table 4 presents the details of the injectability data.

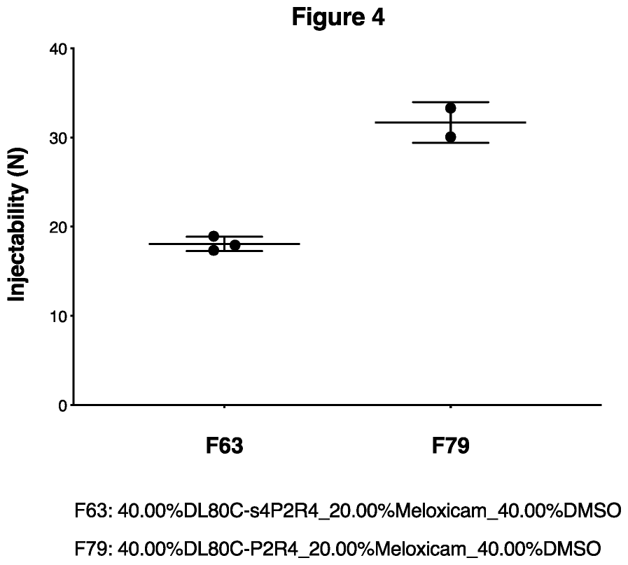

FIG. 5 is a graph showing the percentage in vitro cumulative release of meloxicam over time from F88. Formulation F88 (□) contains 50.00% of DL80C-s3P0.45R6 star-shaped copolymer with 10.00% of active pharmaceutical ingredient (API) and 40.00% of DMSO. In vitro release tests have been conducted according to set up 2 in table 3, example 3. The specific block copolymer formulations are set forth in Table 1 below.

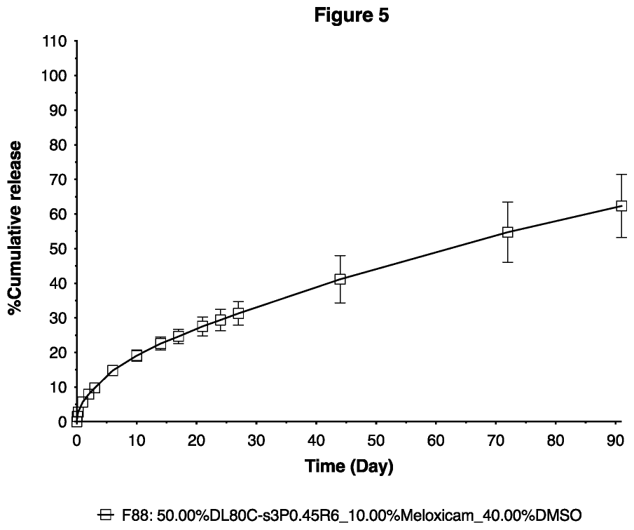

Results demonstrate that star-shaped copolymer-based formulation leads to a sustained release of the drug for up to at least 91 days.

FIG. 6 presents the percentage in vitro cumulative release of meloxicam over time from three different formulations. Formulation F85 (◯) containing 30.00% of DL80C-s4P2R4 star-shaped copolymer with 10.00% of active pharmaceutical ingredient (API) and 60.00% of DMSO, F74 (□) containing 40.00% of DL80C-s4P2R4 star-shaped copolymer with 10.00% of active pharmaceutical ingredient (API) and 50.00% of DMSO and formulation F86 (Δ) containing 50.00% of DL80C-s4P2R4 star-shaped copolymer with 10.00% of active pharmaceutical ingredient (API) and 40.00% of DMSO. In vitro release tests have been conducted according to set up 2 in table 3, example 3. The specific block copolymer formulations are set forth in Table 1 below.

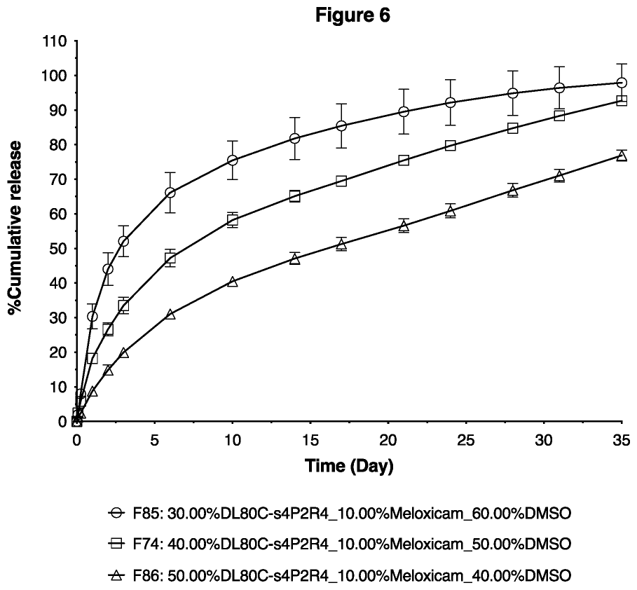

Data show that an increase of the star-shaped copolymer content leads to a decrease in the release rate. Indeed, formulation F86 shows slower release kinetics compared to F74 and F85. Similarly, formulation F74 shows slower release kinetics compared to F85.

FIG. 7 shows the percentage total in vitro cumulative release of meloxicam over time from two different formulations. Formulation F74 (□) containing 40.00% of DL80C-s4P2R4 star-shaped copolymer with 10.00% of active pharmaceutical ingredient (API) and 50.00% of DMSO; and formulation F76 (∇) containing 40.00% of DL80C-s4P2R6 star-shaped copolymer with 10.00% of active pharmaceutical ingredient (API) and 50.00% of DMSO. In vitro release tests have been conducted according to set up 2 in table 3, example 3. The specific block copolymer formulations are set forth in Table 2.

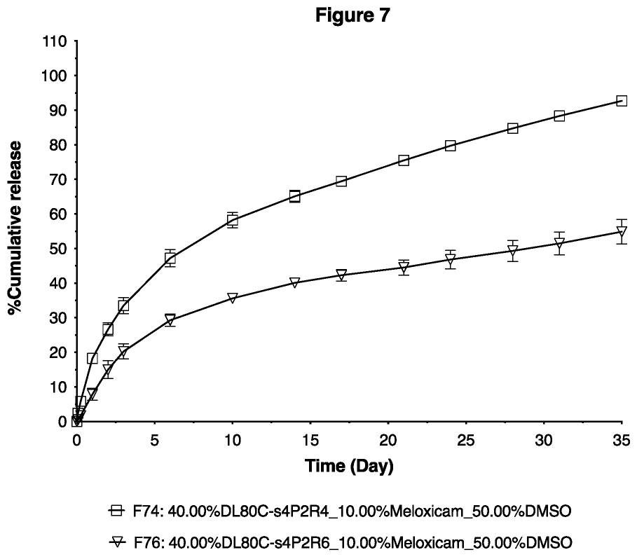

Data show that an increase of the PCLA chain length (molecular weight) within the star-shaped copolymer leads to the modulation of release kinetics in formulations with the same copolymer content. Formulation F76 shows slower release kinetics than F74.

FIG. 8 is a graph showing the percentage in vitro cumulative release of meloxicam over time from F89. Formulation F89 (⊗) contains 44.40% of DL80C-s4P5R4 star-shaped copolymer with 10.00% of active pharmaceutical ingredient (API) and 45.60% of DMSO. In vitro release tests have been conducted according to set up 2 in table 3, example 3. The specific block copolymer formulations are set forth in Table 2 below.

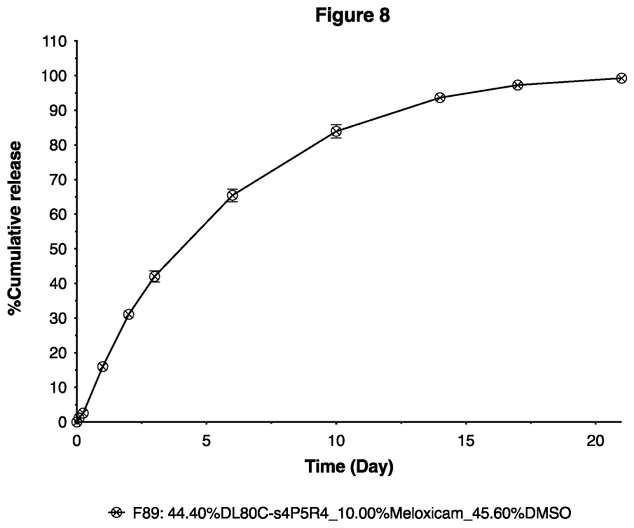

Results demonstrate that star-shaped copolymer-based formulation leads to a sustained release of the drug for up to at least 21 days.

FIG. 9 shows the percentage total in vitro cumulative release of meloxicam over time from two different formulations. Formulation F73 (◯) containing 40.00% of DL50C-s4P2R4 star-shaped copolymer with 10.00% of active pharmaceutical ingredient (API) and 50.00% of DMSO and formulation F75 (Δ) containing 40.00% of DL90C-s4P2R4 star-shaped copolymer with 10.00% of active pharmaceutical ingredient (API) and 50.00% of DMSO. In vitro release tests have been conducted according to set up 2 in table 3, example 3. The specific block copolymer formulations are set forth in Table 2.

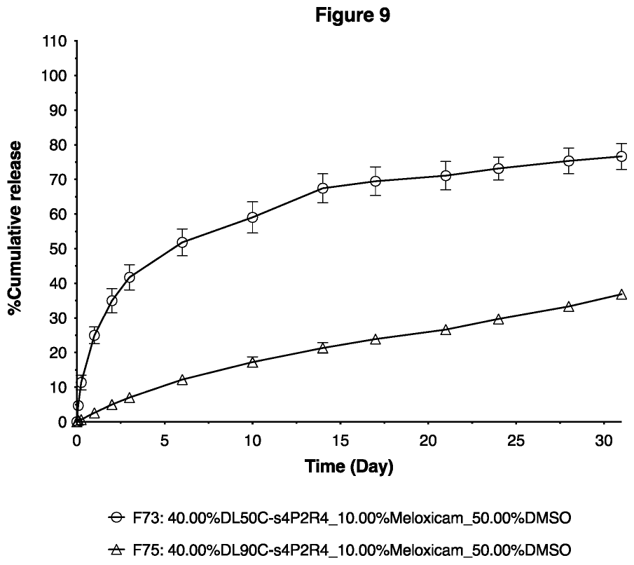

Data show that a modification of the LA/CL molar ratio within the star-shaped copolymer leads to the modulation of release kinetics in formulations with the same copolymer content. Formulation F75 shows slower release kinetics than F73.

FIG. 10 shows the percentage in vitro cumulative release of meloxicam over time from two different formulations: Formulation F91 (Δ) containing 40.00% of DL80C-P2R4 triblock copolymer with 10.00% of active pharmaceutical ingredient (API) and 50.00% of NMP and formulation F90 (∇) containing 40.00% of DL80C-s4P2R4 star-shaped copolymer with 10.00% of active pharmaceutical ingredient (API) and 50.00% of NMP. In vitro release tests have been conducted according to set up 2 in table 3, example 3. The specific block copolymer formulations are set forth in Table 2.

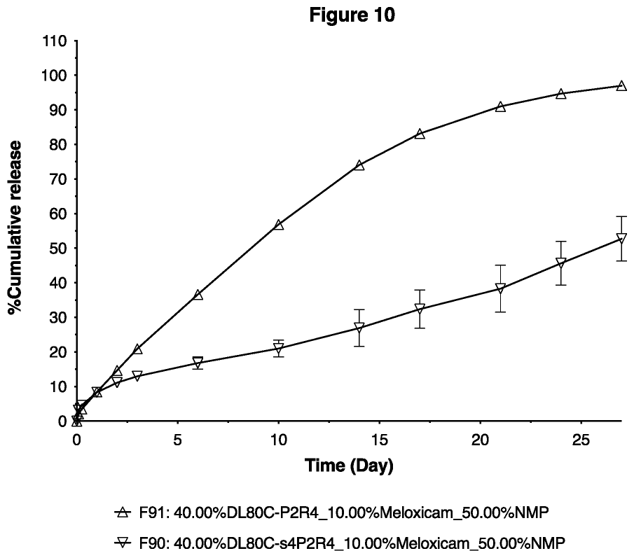

Results indicate that star-shaped copolymers-based formulation leads to a slower release kinetics compared to linear copolymer-based formulations with an identical copolymer content and a comparable molecular weight. Indeed, formulation F90 shows slower release kinetics compared to F91.

FIG. 11 shows the percentage in vitro cumulative release of Tamsulosin over time from two different formulations: Formulation F94 (◯) containing 40.00% of DL80C-P2R4 triblock copolymer with 14.40% of active pharmaceutical ingredient (API) and 45.60% of DMSO and formulation F92 (□) containing 40.00% of DL80C-s4P2R4 star-shaped copolymer with 14.40% of active pharmaceutical ingredient (API) and 45.60% of DMSO. In vitro release tests have been conducted according to set up 1 in table 3, example 3. The specific block copolymer formulations are set forth in Table 2.

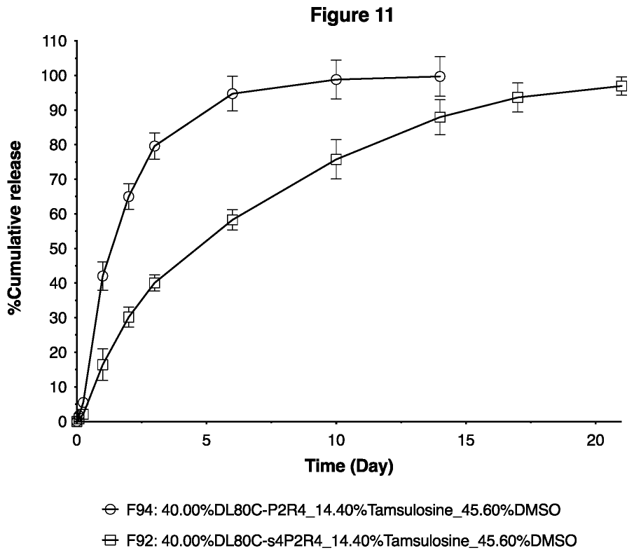

Results indicate that a star-shaped copolymer-based formulation exhibits slower release kinetics compared to a linear copolymer-based formulation for a comparable molecular weight and an identical total copolymer content. Indeed, formulation F92 shows slower release kinetics than formulation F94.

FIG. 12 shows the percentage total in vitro cumulative release of Tamsulosin over time from two different formulations. Formulation F92 (◯) containing 40.00% of DL80C-s4P2R4 star-shaped copolymer with 14.40% of active pharmaceutical ingredient (API) and 45.60% of DMSO; and formulation F93 (□) containing 40.00% of DL80C-s4P2R6 star-shaped copolymer with 14.40% of active pharmaceutical ingredient (API) and 45.60% of DMSO. In vitro release tests have been conducted according to set up 1 in table 3, example 3. The specific block copolymer formulations are set forth in Table 2.

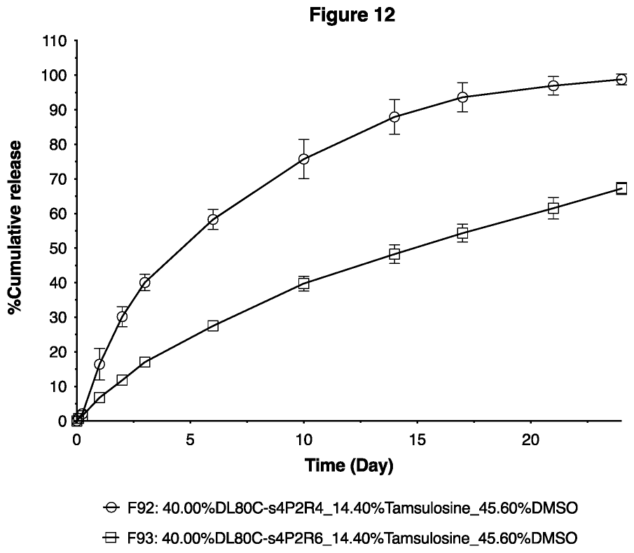

Data show that an increase of the PCLA chain length (molecular weight) within the star-shaped copolymer leads to the modulation of release kinetics in formulations with the same copolymer content. Formulation F93 shows slower release kinetics than F92.

FIG. 13 is a graph showing the total active moiety plasma concentration expressed in nanogram per milliliter of meloxicam over time from F76. Formulation F76 (⊗) contains 40.00% of DL80C-s4P2R6 star-shaped copolymer with 10.00% of active pharmaceutical ingredient (API) and 50.00% of DMSO. In vivo release tests have been conducted according to set up disclosed in example 6.

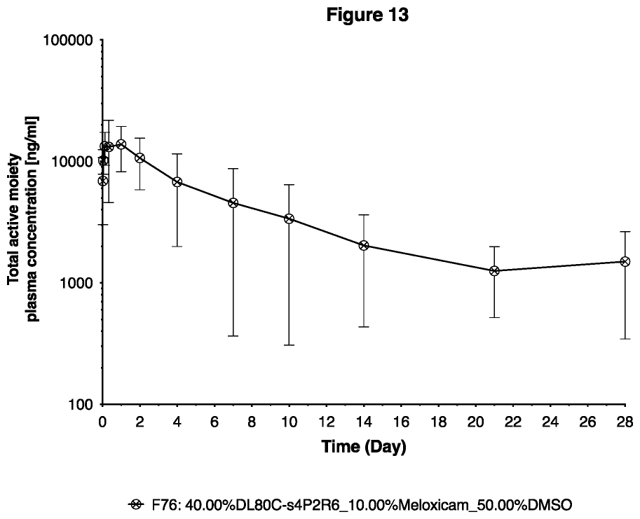

Results demonstrate that star-shaped a copolymer-based formulation leads to a sustained release of the drug in vivo for at least up to 28 days.

### EXAMPLES

### Example 1: Materials

### Star-Shaped Block Copolymers

Set out below is a generic reaction scheme to obtain a multi-branched PEG-PCLA copolymer as used in the pharmaceutical compositions of the invention. The letters m and n describe the number of repetitive units in each PEG and polyester block respectively. The letter q describes the relative LA/CL molar content. Considering the synthetic pathway and experimental conditions, it is assumed that the multi-arm polymers are symmetrical, and each arm displays the same structure and composition. It will be understood that although in scheme 1 below, a 3-arm PEG derivative is used; an analogous reaction scheme can be used with a multi-branched PEG having a different number of PEG arms.

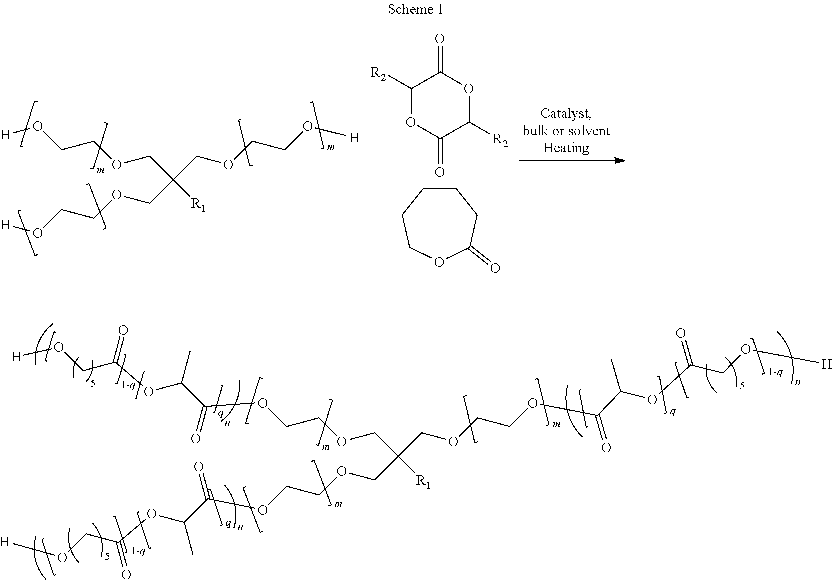

Multi-branched block copolymers are synthesized by ring-opening polymerization of D,L-lactide and ε-caprolactone initiated by multi-branched polyethers referred to as multi-branched PEGs or star PEGs. D,L lactide and ε-caprolactone monomers are available in a wide number of suppliers, typically they can be purchased respectively from Corbion (Diemen, Netherlands) and Alfa Aesar (Ward Hill, MA, USA). The ring-opening polymerization of lactones is carried out in an inert atmosphere, e.g. nitrogen, argon, either by conventional heating or via microwave irradiation. Various molecular weights and architectures of star PEG macroinitiator, i.e. 3-arm, 4-arm PEG-OH as shown in scheme 1, are commercially available from several suppliers, such as Creative PEGWorks (Durham, NC, USA), Jenkem (Plano, TX, USA) or Sigma-Aldrich (Saint-Louis, MO, USA). Alternatively, multi-branched PEG can be formed by the reaction of ethylene oxide with a polyol.

In a general procedure for conventional heating, the multi-branched PEG and 150-250 ppm of catalyst, corresponding to 0.15-6.50 mol % per hydroxyl group, are introduced in a round-bottom flask in inert atmosphere. Subsequently, an appropriate amount of lactone based on the targeted R and monomers ratios is added to the mixture at 80° C. The reaction mixture is subjected to one cycle of inert atmosphere/vacuum and is then heated at 130° C. overnight. In a general procedure for microwave-assisted polymerization, all the reactants in appropriate amount, i.e. multi-branched PEG, catalyst and lactones, are added at the same time in the vessel and heated at 200° C. for 25 minutes.

Independently of the type of heating, the obtained copolymer is cooled down at room temperature. The crude polymer is solubilised and filtered through activated charcoal to remove the catalyst, and precipitated to remove any unreacted monomers and oligomers. Then, the pure polymer is dried under vacuum overnight.

Star-shaped block copolymers are characterized after reaction and purification by ¹H NMR, DSC and GPC to ensure that the targeted polymer characteristics are reached.

¹H NMR spectra are recorded by an external company according to their standard procedure on a Bruker Avance 300 MHz spectrometer into an appropriate deuterated solvent, e.g. deuterated chloroform (CDCl₃). Characteristics, such as monomer(s) conversion, monomer(s) ratio and R ratio, among others, are calculated from the characteristic peak integration.

Gel permeation chromatography (GPC) measurements are carried out on a gel permeation chromatography triple detector array (GPC-TDA) apparatus supplied by Malvern. 2 mL of THF solution at 15 mg/mL of polymer is prepared, filtered and put into 1.5 mL vials with screw caps for analysis. After the determination of the do/dc value for each polymer, 100 of polymer solution are injected in the GPC system. Characteristics, such as Mₙ and dispersities (Ð), intrinsic viscosity, among others, are considered and the mean value obtained from the injections is summarized in table 1 below.

DSC assays are performed using a Mettler Toledo DSC3+ calibrated with indium standards. In a typical experiment, the sample (generally 5-10 mg, weighted and sealed into a 40 aluminium crucible) was cooled down to −80° C. and an initial ramp up to 100° C. was performed to erase its thermal history. The polymer was then cooled to −80° C. and a second heating scan up to 200° C. was carried out to determine the thermal transitions. All the scans were performed at a heating rate of 10° C. min⁻¹ and the experiments were carried out under a N₂ flow (50 mL min⁻¹). All the heating and cooling ramps were followed by a 10-minute isothermal step to equilibrate the sample. The T_{g} was taken as the temperature at the half-height point of the heat flow change. Each experiment was performed in duplicates to give data confidence. The error on the measured T_{g}s was typically 0.2-0.5° C. and the average values (rounded to the closest integer) are reported.

TABLE 1

| | | | | | | | | | | ¹H-NMR^{b} | | | |
|---|---|---|---|---|---|---|---|---|---|---|---|---|---|
| | | | | R ratio^{th} | | GPC-TDA^{a} | | | | | R ratio | | |
| | | | LA | [(LA + | | | | | IV | | [(LA + | LA | DSC |
| | | Mₙ^{PEG} | content^{th} | CL)/ | Mₙ^{th} | Mₙ | | Mₚ | (dL · | Mₙ | CL)/ | content | T_{g} |
| Product | Structure | (kDa) | (%) | EO] | (kDa) | (kDa) | Ð | (kDa) | g⁻¹) | (kDa) | EO] | (%) | (° C.) |
| DL30C-s4P2R4 | Branched | 2.00 | 30 | 4 | 20.4 | 7.4 | 2.0 | 15.3 | 0.2183 | 17.9 | 3.5 | 30.9 | −51 |
| DL50C-s4P2R4 | Branched | 2.00 | 50 | 4 | 18.9 | 11.6 | 1.3 | 15.2 | 0.2026 | 16.9 | 3.6 | 52.0 | −37 |
| DL80C-s3P0.45R6 | Branched | 0.45 | 80 | 6 | 5.4 | 5.1 | 1.1 | 5.1 | 0.0981 | 3.2 | 6.4 | 71.4 | −20 |
| DL80C-s4P2R4 | Branched | 2.00 | 80 | 4 | 16.6 | 10.2 | 1.4 | 14.7 | 0.1670 | 16.1 | 3.8 | 75.0 | −17 |
| DL80C-s4P2R6 | Branched | 2.00 | 80 | 6 | 23.9 | 10.6 | 1.7 | 19.6 | 0.1700 | 21.7 | 5.5 | 84.1 | −3 |
| DL80C-s4P5R4 | Branched | 5.00 | 80 | 4 | 42.5 | 59.9 | 1.7 | 119.1 | 0.2632 | 37.6 | 3.5 | 76.9 | −13 |
| DL90C-s4P2R4 | Branched | 2.00 | 90 | 4 | 15.8 | 10.8 | 1.3 | 13.7 | 0.1587 | 14.5 | 3.7 | 93.6 | 9 |
| DL80C-P2R4 | Linear | 2.00 | 80 | 4 | 16.6 | 9.9 | 1.2 | 11.5 | 0.1947 | 14.6 | 3.6 | 86.1 | −3 |

^{th}Theoretical;
^{a}In THF (15 mg mL⁻¹, 30° C.) at 1 mL min⁻¹;
^{b}In CDCl₃ (5 mg mL⁻¹);
^{c}From 2^{nd} heating scan in N₂ (10° C. min⁻¹).

### Linear Block Copolymers (Comparative)

The multi-branched copolymers of the invention were compared to linear diblock and triblock copolymers.

Comparative triblock copolymers have the formula:

Av-Bw-Ax
wherein A is PCLA and B is polyethylene glycol and v and x are the number of repeat units ranging from 1 to 3,000 and w is the number of repeat units ranging from 3 to 300 and v=x or v≠x.

Comparative diblock copolymers have the formula:

C_{y}-A_{z}
wherein A is PCLA and C is methoxy PEG and y and z are the number of repeat units with y ranging from 2 to 250 and z ranging from 1 to 3,000.

Further description of the linear triblock and diblock copolymers used as comparative examples can be found in WO2012/090070A1, WO2019016233A1, WO2019016234A1, and WO2019016236A1 incorporated by reference herein.

Linear block copolymers are synthesized by ring-opening polymerization of a mixture of D,L-lactide and ε-caprolactone initiated by PEG (triblock copolymer) or methoxy-PEG (diblock copolymer). The ring-opening polymerization of lactones is carried out in inert atmosphere, e.g. nitrogen, argon, either by conventional heating or via microwave irradiation.

In a general procedure for conventional heating, the PEG or methoxy-PEG and 150-250 ppm of catalyst, corresponding to 0.15-6.50 mol % per hydroxyl group, are introduced in a round-bottom flask in inert atmosphere. Subsequently, an appropriate amount of lactones based on the targeted R and monomers ratios is added to the mixture at 80° C. The reaction mixture is subjected to one cycle of inert atmosphere/vacuum and is then heated at 130° C. overnight.

In a general procedure for microwave-assisted polymerization, all the reactants in an appropriate amount, i.e. PEG or methoxy-PEG, catalyst and lactones, are added at the same time in the vessel and heated at 200° C. for 25 minutes.

Independently of the type of heating, the obtained copolymer is cooled down at room temperature. The crude polymer is solubilised and filtered through activated charcoal to remove the catalyst, and precipitated to remove any unreacted monomers and oligomers. Then, the pure polymer is dried under vacuum overnight.

Linear block copolymers are characterized after reaction and purification by ¹H NMR, GPC and DSC to ensure that the targeted polymer characteristics are reached. Same methods than for star-shaped block copolymers were used for characterization.

### Example 2: Analysis of Soluble Fraction of Star-Shaped Copolymers in Water

Water solubility tests were performed to determine the soluble fraction of star-shaped copolymers in water.

Water solubility analysis consisted of the following steps:
Empty 20 mL vials were weighed (1). 500 mg of copolymer was weighed and added to the corresponding vial. 5 mL of ultra-pure water was added to each vial. Vials were incubated for 2 h at 37° C. while vortexing. Visual observations were carried out and pictures were taken. The vials (1) were then centrifuged for 10 mins at 3000 rpm. A 10 mL glass vial (2) was weighed. The supernatant of (1) was transferred into (2) and masses were recorded. The vial (2) was placed at −80° C. overnight and then placed in the freeze dryer for 22 h. Water solubility was determined after drying and weighing the remaining dried copolymer in the vial (2). The amount of dissolved copolymer was determined as the difference of weight of the empty vial and the lyophilized one. The analysis of water solubility was performed in a single analysis.

Results show water solubility values of 0.13 mg/mL and 0.34 mg/mL for DL80C-s4P2R4 and DL80C-s4P5R4 respectively.

### Example 3: In Vitro Release Tests

Set-up 1 detailed procedure for meloxicam:

### Formulation Preparation

In an empty and tared 3 mL glass vial, required copolymer amounts were weighed. The glass vial was tared again. An accurate DMSO mass was added using a Pasteur pipette. Vehicles (copolymer+solvent) were then placed on a roller mixer at room temperature (RT) for 6 to 7 hours until complete copolymer dissolution. Glass vials were then tared, and the required API amount was weighed. The formulations were then placed overnight at room temperature on a roller mixer.

### Determination of Soluble Fraction (SF) of API in Suspension-Formulations

Soluble fraction determination tests were performed in parallel with in vitro release (IVR) to determine the exact API percentage solubilized in suspension-formulations. This test was made in triplicate. 500 μL of formulation was withdrawn from the corresponding glass vial previously vortexed, into a 0.5 mL Codan syringe. The formulation was injected onto a 1.5 mL filtered Eppendorf tube and centrifugated for 5 min at 13200 rpm at RT. After centrifugation, 50 μL of supernatant was withdrawn into a 0.5 mL Codan syringe. The syringe was cleaned, tared and the formulation was directly injected from the syringe without needle, into a 50 mL empty Falcon tube. The empty syringe was weighed, and the exact supernatant mass that was injected into the Falcon tube was recorded. Supernatant was dissolved in 15 mL of HPLC-grade acetonitrile and the solution was vortexed. After complete dissolution, 5 mL of ultra-pure water were added, and the solution was vortexed. 1 mL of sample was filtered through a 0.45 μm PTFE Phenomenex filter into a 1.5 mL HPLC vial. API soluble fraction was determined using UPLC. The amount of meloxicam in solution was calculated from a calibration curve where the concentration of meloxicam ranges between 0 and 160 μg/ml.

### In Vitro Release Set Up

50 μL of formulation were withdrawn from the corresponding glass vial previously vortexed, into a 0.5 mL Codan syringe. The syringe was cleaned, tared and the formulation was directly injected from the syringe without needle, into a 50 mL prefilled glass vial containing 20 mL of release buffer. The buffer used is phosphate-buffered saline (PBS) pH 7.4, which was 137 mM sodium chloride, 2.7 mM potassium chloride, 10 mM disodium phosphate, 1.8 mM monopotassium phosphate and 0.1% sodium azide. Upon injection, the solvent diffused away from the formulation and the remaining polymer forms an in situ depot within the aqueous environment. Once precipitation and depot formation had occurred, the depot was cut from the syringe using scissors. The syringe was weighed back to determine the accurate depot mass.

The meloxicam incorporated into the polymer solution was encapsulated within the polymer matrix as it solidifies.

Once all depots were formed, glass vials were maintained under constant shaking at 180 rpm (Unimax 1010 apparatus, Heidolph) at 37° C.

IVR was analyzed following the steps detailed below:

### IVR Sampling and Preparation of IVR Samples for API Quantification

At each desired time point, a sufficient amount of buffer was withdrawn for analysis from the 50 mL glass vial before total buffer refreshment. 1 mL of each sample was filtered through a 0.2 μm hydrophilic filter into a 1 mL HPLC glass vial. The rest of the medium was discarded and 20 mL of fresh buffer were added to the glass vial. Sink conditions were maintained during the full duration of the study. API contents in released buffer were determined using UPLC. The amount of meloxicam released from the formulation was calculated from a calibration curve where the concentration of meloxicam ranges between 0 and 160 μg/ml.

Some parameters, for example the mass of formulation, the buffer volume may be adapted depending on the studied API, its solubility in buffer and its targeted dose and release duration. Set-up with different parameters is presented in Table 3 below.

All of the studied formulations are presented in Table 2 below.

TABLE 2

| | | | | Copolymer | | | | | | | | |
|---|---|---|---|---|---|---|---|---|---|---|---|---|
| | | | | | | | | | Ratio | | | |
| | API | | | | | LA | CL | PEG | [(LA + | | Solvent | |
| Formulation | | % | SF | | | content | content | size | CL)/ | % | | % |
| Number | Name | (w/w) | (%) | Code | Structure | (%) | (%) | (kDa) | EO] | (w/w) | Name | (w/w) |
| 62 | Meloxicam | 20.00 | 10 | DL90C- | Branched | 90 | 10 | 2.00 | 4 | 40.00 | DMSO | 40.00 |
| | | | | s4P2R4 | | | | | | | | |
| 63 | Meloxicam | 20.00 | 14 | DL80C- | Branched | 80 | 20 | 2.00 | 4 | 40.00 | DMSO | 40.00 |
| | | | | s4P2R4 | | | | | | | | |
| 64 | Meloxicam | 20.00 | 14 | DL30C- | Branched | 30 | 70 | 2.00 | 4 | 40.00 | DMSO | 40.00 |
| | | | | s4P2R4 | | | | | | | | |
| 71 | Meloxicam | 2.00 | 0 | DL90C- | Branched | 90 | 10 | 2.00 | 4 | 40.00 | DMSO | 58.00 |
| | | | | s4P2R4 | | | | | | | | |
| 73 | Meloxicam | 10.00 | 36 | DL50C- | Branched | 50 | 50 | 2.00 | 4 | 40.00 | DMSO | 58.00 |
| | | | | s4P2R4 | | | | | | | | |
| 74 | Meloxicam | 10.00 | 31 | DL80C- | Branched | 80 | 20 | 2.00 | 4 | 40.00 | DMSO | 50.00 |
| | | | | s4P2R4 | | | | | | | | |
| 75 | Meloxicam | 10.00 | 27 | DL90C- | Branched | 90 | 10 | 2.00 | 4 | 40.00 | DMSO | 50.00 |
| | | | | s4P2R4 | | | | | | | | |
| 76 | Meloxicam | 10.00 | 29 | DL80C- | Branched | 80 | 20 | 2.00 | 6 | 40.00 | DMSO | 50.00 |
| | | | | s4P2R6 | | | | | | | | |
| 79 | Meloxicam | 20.00 | 12 | DL80C- | Linear | 80 | 20 | 2.00 | 4 | 40.00 | DMSO | 40.00 |
| | | | | P2R4 | | | | | | | | |
| 85 | Meloxicam | 10.00 | 38 | DL80C- | Branched | 80 | 20 | 2.00 | 4 | 30.00 | DMSO | 60.00 |
| | | | | s4P2R4 | | | | | | | | |
| 86 | Meloxicam | 10.00 | 23 | DL80C- | Branched | 80 | 20 | 2.00 | 4 | 50.00 | DMSO | 40.00 |
| | | | | s4P2R4 | | | | | | | | |
| 88 | Meloxicam | 10.00 | 21 | DL80C- | Branched | 80 | 20 | 0.45 | 6 | 50.00 | DMSO | 48.00 |
| | | | | s3P0.45R6 | | | | | | | | |
| 89 | Meloxicam | 10.00 | 22 | DL80C- | Branched | 80 | 20 | 5.00 | 4 | 44.40 | DMSO | 45.60 |
| | | | | s4P5R4 | | | | | | | | |
| 90 | Meloxicam | 10.00 | 41 | DL80C- | Branched | 80 | 20 | 2.00 | 4 | 40.00 | NMP | 50.00 |
| | | | | S4P2R4 | | | | | | | | |
| 91 | Meloxicam | 10.00 | 37 | DL80C- | Linear | 80 | 20 | 2.00 | 4 | 40.00 | NMP | 50.00 |
| | | | | P2R4 | | | | | | | | |
| 92 | Tamsulosin | 14.40 | 33 | DL80C- | Branched | 80 | 20 | 2.00 | 4 | 40.00 | DMSO | 45.60 |
| | | | | s4P2R4 | | | | | | | | |
| 93 | Tamsulosin | 14.40 | 34 | DL80C- | Branched | 80 | 20 | 2.00 | 6 | 40.00 | DMSO | 45.60 |
| | | | | s4P2R6 | | | | | | | | |
| 94 | Tamsulosin | 14.40 | 34 | DL80C- | Linear | 80 | 20 | 2.00 | 4 | 40.00 | DMSO | 45.60 |
| | | | | P2R4 | | | | | | | | |

TABLE 3

| IVR Set-up | Depot formation | | | Buffer | |
|---|---|---|---|---|---|
| Number | Procedure | Injected mass (mg) | Syringe use | Type | Volume (mL) |
| 1 | Injected in the medium | 60 | 0.5 mL Codan syringe | PBS-1X | 20 |
| | from the syringe without | | | | |
| | needle | | | | |
| 2 | Injected in the medium | 60 | 0.5 mL Codan syringe | PBS-1X | 40 |
| | from the syringe without | | | | |
| | needle | | | | |

### Example 4: Injectability

The objective of this experiment was to assess the potential impact of using star-shaped copolymers on the injectability of the vehicles and/or formulations by comparing the values to these vehicles and/or formulations to each other and to analogue linear copolymers.

Injectability analyses were performed using a Lloyd Instruments FT plus texturometer following the procedure described below:

Formulations or vehicle were vortexed for 15 seconds. 500 μL of formulation were withdrawn using a 1 mL Codan syringe without needle. Air bubbles were removed to avoid any interference during the injectability measurement. A 23 G 1″ Terumo needle was then mounted on the syringe, for vehicles or formulations respectively. The syringe was placed onto the texturometer. The flow rate was fixed at 1 mL/min. The speed rate was fixed at 56.3 mm/min. Injection of the formulation started at fixed speed. The injection device (i.e. syringe+needle) was changed for each replicate.

The average force in Newton (N) necessary to inject each replicate was calculated using texturometer software. Using the set up described above, the inventors defined 20 N as the maximum value for a formulation that can be easily injected by hand.

TABLE 4

| | | | Copolymer | | | | | | | | | |
|---|---|---|---|---|---|---|---|---|---|---|---|---|
| | | | | | | Ratio | | | | | | |
| | API | | | | PEG | [(LA + | | Solvent | | Injectability | | |
| Sample | | % | | | size | CL)/ | % | | % | Replicate | Force | |
| Number | Name | (w/w) | Code | Structure | (kDa) | EO] | (w/w) | Name | (w/w) | number | (N) | S [symbol] |
| V1 | NA | NA | DL50C-s4P2R4 | Branched | 2.00 | 4 | 40.00 | DMSO | 60.00 | 3 | 6.3 | 0.6 |
| V2 | NA | NA | DL80C-s3P0.45R6 | Branched | 0.45 | 6 | 40.00 | DMSO | 60.00 | 3 | 1.9 | 0.1 |
| V3 | NA | NA | DL80C-s4P2R4 | Branched | 2.00 | 4 | 40.00 | DMSO | 60.00 | 3 | 4.2 | 0.1 |
| V4 | NA | NA | DL80C-s4P2R6 | Branched | 2.00 | 6 | 40.00 | DMSO | 60.00 | 3 | 5.8 | 0.3 |
| V5 | NA | NA | DL80C-s4P5R4 | Branched | 5.00 | 4 | 40.00 | DMSO | 60.00 | 3 | 7.7 | 0.5 |
| V6 | NA | NA | DL80C-P2R4 | Linear | 2.00 | 4 | 40.00 | DMSO | 60.00 | 3 | 5.5 | 0.1 |
| V7 | NA | NA | DL90C-s4P2R4 | Branched | 2.00 | 4 | 40.00 | DMSO | 60.00 | 3 | 4.6 | 0.2 |
| V8 | NA | NA | DL50C-s4P2R4 | Branched | 2.00 | 4 | 40.00 | Tripropionin | 60.00 | 3 | 16.8 | 2.4 |
| V9 | NA | NA | DL80C-s3P0.45R6 | Branched | 0.45 | 6 | 40.00 | Tripropionin | 60.00 | 3 | 4.9 | 0.3 |
| V10 | NA | NA | DL80C-s4P2R6 | Branched | 2.00 | 6 | 40.00 | Tripropionin | 60.00 | 3 | 19.2 | 0. [symbol] |
| V11 | NA | NA | DL80C-s4P5R4 | Branched | 5.00 | 4 | 40.00 | Tripropionin | 60.00 | 3 | 13.4 | 2. [symbol] |
| V12 | NA | NA | DL80C-P2R4 | Linear | 2.00 | 4 | 40.00 | Tripropionin | 60.00 | 3 | 19.7 | 0. [symbol] |
| V13 | NA | NA | DL90C-s4P2R4 | Branched | 2.00 | 4 | 40.00 | Tripropionin | 60.00 | 3 | 20.9 | 1.5 |
| F63 | Meloxicam | 20.00 | DL80C-s4P2R4 | Branched | 2.00 | 4 | 40.00 | DMSO | 40.00 | 3 | 12.2 | 0. [symbol] |
| F64 | Meloxicam | 20.00 | DL30C-s4P2R4 | Branched | 2.00 | 4 | 40.00 | DMSO | 40.00 | 3 | 22.0 | 0. [symbol] |
| F73 | Meloxicam | 10.00 | DL50C-s4P2R4 | Branched | 2.00 | 4 | 40.00 | DMSO | 50.00 | 3 | 11.8 | 0.8 |
| F74 | Meloxicam | 10.00 | DL80C-s4P2R4 | Branched | 2.00 | 4 | 40.00 | DMSO | 50.00 | 3 | 6.1 | 0.1 |
| F75 | Meloxicam | 10.00 | DL90C-s4P2R4 | Branched | 2.00 | 4 | 40.00 | DMSO | 50.00 | 3 | 10.0 | 0.6 |
| F76 | Meloxicam | 10.00 | DL80C-s4P2R6 | Branched | 2.00 | 6 | 40.00 | DMSO | 50.00 | 3 | 10.8 | 0.8 |
| F79 | Meloxicam | 20.00 | DL80C-P2R4 | Linear | 2.00 | 4 | 40.00 | DMSO | 40.00 | 2 | 31.7 | 2.3 |
| F88 | Meloxicam | 10.00 | DL80C-s3P0.45R6 | Branched | 0.45 | 6 | 50.00 | DMSO | 40.00 | 3 | 6.8 | 0.3 |

[symbol]

indicates data missing or illegible when filed

### Example 5: Dynamic Viscosity Analysis

Dynamic viscosity analysis was performed using an Anton Paar Rheometer equipped with cone plate measuring system, with the following analytical conditions:
Measuring system: cone plate of 50 mm diameter and cone angle of 1 degree (CP50). CP50 is better appropriate to obtain precise values for formulations showing low viscosity.
Working range: from 10 to 1000 mPa·s.
Temperature controlled at 25° C.
Amount of vehicle: 0.7 mL.

The formulation or vehicle was vortexed for 10 s before analysis. The appropriate amount of sample was placed at the centre of the thermo-regulated measuring plate using a spatula. The measuring system was lowered down and a 0.104 mm gap was left between the measuring system and the measuring plate. Twenty-one viscosity measurements points were determined across the 10 to 1000 s⁻¹ shear rate (10 points per decade). For suspensions, 60 s rest time was set at the measuring position prior the analysis followed by a pre-shearing at 1000 s⁻¹ for 30 s. Viscosity data correspond to those calculated at a shear rate of 100 s⁻¹, which is an average value of the curve plateau. The dynamic viscosity analyses were performed in duplicates when sufficient amount was available.

TABLE 5

| | | | Copolymer | | | | | | | | | |
|---|---|---|---|---|---|---|---|---|---|---|---|---|
| | | | | | | Ratio | | | | Viscosity | | |
| | API | | | | PEG | [(LA + | | Solvent | | | Dynamic | |
| Sample | | % | | | size | CL)/ | % | | % | Replicate | viscosity | |
| Number | Name | (w/w) | Code | Structure | (kDa) | EO] | (w/w) | Name | (w/w) | number | (mP · s) | S [symbol] |
| V1 | NA | NA | DL50C-s4P2R4 | Branched | 2.00 | 4 | 40.00 | DMSO | 60.00 | 2 | 363.2 | 4.0 |
| V2 | NA | NA | DL80C-s3P0.45R6 | Branched | 0.45 | 6 | 40.00 | DMSO | 60.00 | 2 | 67.8 | 0.4 |
| V3 | NA | NA | DL80C-s4P2R4 | Branched | 2.00 | 4 | 40.00 | DMSO | 60.00 | 2 | 262.3 | 3.8 |
| V4 | NA | NA | DL80C-s4P2R6 | Branched | 2.00 | 6 | 40.00 | DMSO | 60.00 | 2 | 369.3 | 1.4 |
| V5 | NA | NA | DL80C-s4P5R4 | Branched | 5.00 | 4 | 40.00 | DMSO | 60.00 | 1 | 494.0 | NA |
| V6 | NA | NA | DL80C-P2R4 | Linear | 2.00 | 4 | 40.00 | DMSO | 60.00 | 1 | 346.7 | NA |
| V7 | NA | NA | DL90C-s4P2R4 | Branched | 2.00 | 4 | 40.00 | DMSO | 60.00 | 2 | 246.9 | 2.2 |
| V8 | NA | NA | DL50C-s4P2R4 | Branched | 2.00 | 4 | 40.00 | Tripropionin | 60.00 | 1 | 1259.3 | NA |
| V9 | NA | NA | DL80C-s3P0.45R6 | Branched | 0.45 | 6 | 40.00 | Tripropionin | 60.00 | 2 | 260.3 | 1.8 |
| V10 | NA | NA | DL80C-s4P2R6 | Branched | 2.00 | 6 | 40.00 | Tripropionin | 60.00 | 1 | 1378.7 | N [symbol] |
| V11 | NA | NA | DL80C-s4P5R4 | Branched | 5.00 | 4 | 40.00 | Tripropionin | 60.00 | 1 | 1385.6 | N [symbol] |
| V12 | NA | NA | DL80C-P2R4 | Linear | 2.00 | 4 | 40.00 | Tripropionin | 60.00 | 1 | 1208.1 | N [symbol] |
| V13 | NA | NA | DL90C-s4P2R4 | Branched | 2.00 | 4 | 40.00 | Tripropionin | 60.00 | 1 | 1360.9 | NA |
| F64 | Meloxicam | 20.00 | DL30C-s4P2R4 | Branched | 2.00 | 4 | 40.00 | DMSO | 40.00 | 2 | 1481.2 | 5. [symbol] |
| 73 | Meloxicam | 10.00 | DL50C-s4P2R4 | Branched | 2.00 | 4 | 40.00 | DMSO | 50.00 | 2 | 940.5 | 45 [symbol] |
| 75 | Meloxicam | 10.00 | DL90C-s4P2R4 | Branched | 2.00 | 4 | 40.00 | DMSO | 50.00 | 2 | 619.5 | 9.7 |
| 76 | Meloxicam | 10.00 | DL80C-s4P2R6 | Branched | 2.00 | 6 | 40.00 | DMSO | 50.00 | 2 | 972.4 | 19.5 |
| 88 | Meloxicam | 10.00 | DL80C-s3P0.45R6 | Branched | 0.45 | 6 | 50.00 | DMSO | 40.00 | 2 | 386.8 | 4.4 |

[symbol]

indicates data missing or illegible when filed

### Example 6: Pharmacokinetic Study

### In Vivo Detailed Set-Up Procedure

A meloxicam formulation was tested in a pharmacokinetic study in male adult rats with a weight between 200 and 250 g. Drug product containing 18.6 mg of meloxicam was subcutaneously administered in the interscapular area of the rats using 1 mL Soft Ject® syringes and 23 G (1″ 0.6×25 mm) Terumo® needles. Injected formulation volumes were fixed to 160 μL. Blood samples were collected into EDTA tubes at different time points: T0.5h, T1h, T3h, T8h, T24h (Day 1), T48h (Day 2), T96h (Day 4), T168h (Day 7), T240h (Day 10), T336h (Day 14),), T504h (Day 21),), T672h (Day 28). Blood samples were centrifuged and the plasma from each time point was retained. The plasma samples were analyzed by LC/MS/MS to quantify meloxicam content.

1. A pharmaceutical composition comprising:
a multi-branched copolymer comprising at least three polyester arms, wherein the polyester is poly(ε-caprolactone-co-lactic acid), attached to a central core which comprises a polyether, and wherein the multi-branched copolymer is substantially insoluble in aqueous solution,
at least one pharmaceutically active ingredient, and
a pharmaceutically acceptable organic solvent in an amount of at least 20% (w/w %) of the total composition.

2. The composition according to claim 1, wherein the molecular weight of the polyether is 10 kDa or less.

3. A pharmaceutical composition comprising:
a multi-branched copolymer comprising at least three polyester arms, wherein the polyester is poly(ε-caprolactone-co-lactic acid), attached to a central core which comprises a polyether, and wherein the molecular weight of the polyether is 10 kDa or less
at least one pharmaceutically active ingredient, and
a pharmaceutically acceptable organic solvent in an amount of at least 20% (w/w %) of the total composition.

4. (canceled)

5. The composition according to claim 3, wherein the multi-branched copolymer has less than 15 mg/mL solubility in aqueous solution.

6. The composition according to claim 5, wherein aqueous solubility is measured at 37° C.

7. (canceled)

8. The composition according to claim 3, wherein the multi-branched copolymer is of formula A(B)ₙ wherein A represents the central core, and B represents the polyester arms, n is an integer of 3.

9. The composition according to claim 3, wherein the central core is a multi-branched polyether which is derivable from poly(ethylene glycol) (PEG) and a polyol.

10. The composition according to claim 9, wherein the polyol comprises at least three hydroxyl groups.

11. The composition according to claim 10, wherein the polyol is a hydrocarbon substituted with 3 to 8 hydroxyl groups.

12. The composition according to claim 9, wherein the polyol further comprises one or more ether groups.

13. The composition according to claim 9, wherein the polyol is pentaerythritol (PE), dipentaerythritol (DPE), trimethylolpropane (TMP), trimethylolmethane, glycerol, hexaglycerol, erythritol, xylitol, di(trimethylolpropane) (diTMP), sorbitol, or inositol.

14. The composition according to claim 9, wherein each branch of the multi-branched polyether has a terminal reactive group capable of reacting with a polyester or monomer or precursor thereof.

15. The composition according to claim 14, wherein the terminal reactive group is a hydroxyl group.

16. The composition according to claim 9, wherein the multi-branched polyether has any of Formulae 1 to 4: 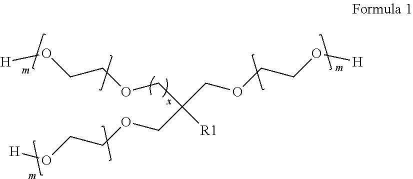
wherein
R₁ is 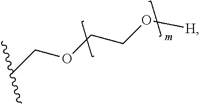
H or alkyl;
x is 0 or 1, and
m is an integer between 2 and 76; 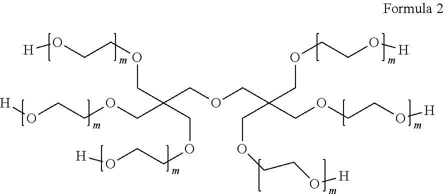
wherein m is an integer between 5 and 40; 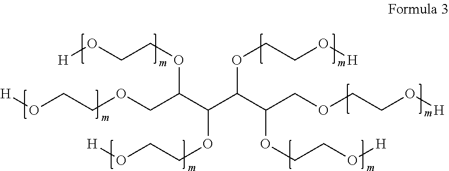
wherein m is an integer between 5 and 40; 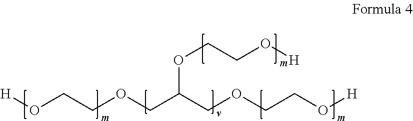
wherein m is an integer between 25 and 30, and v is 6.

17. The composition according to claim 16, wherein the multi-branched polyether has Formula 1, x is 1, and R₁ is alkyl.

18. The composition according to claim 16, wherein the multi-branched polyether has Formula 1, x is 1, and R₁ is

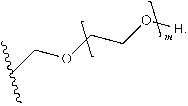

19. The composition according to claim 16, wherein the multi-branched polyether has Formula 1, x is 0, and R₁ is H,

20. The composition according to claim 3, wherein the multi-branched copolymer is obtainable by reacting a multi-branched polyether with D,L-lactide and ε-caprolactone.

21. The composition according to claim 20, where the multi-branched copolymer is obtainable by ring-opening polymerization of the D,L-lactide and ε-caprolactone initiated by the multi-branched polyether.

22. The composition according to claim 21, wherein the number of ester repeat units in each arm is independently in the range of 5 to 230, and wherein the ratio of lactic acid repeat units to hexanoate repeat units is in the range of 25/75 to 99/1.

23. The composition according to claim 3, wherein the multi-branched copolymer has any of Formulae 5 to 8: 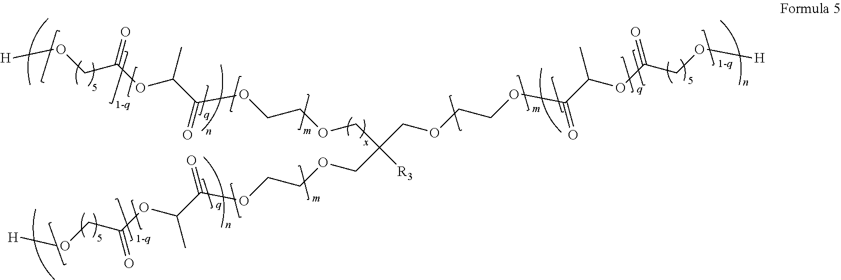
wherein
R₃ is 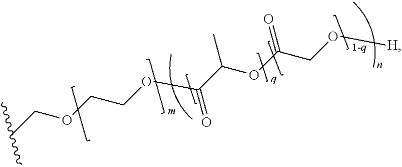
x is 0 or 1,
m is an integer between 2 and 76,
n is an integer between 5 and 230, and
q is between 0.25 and 0.99; 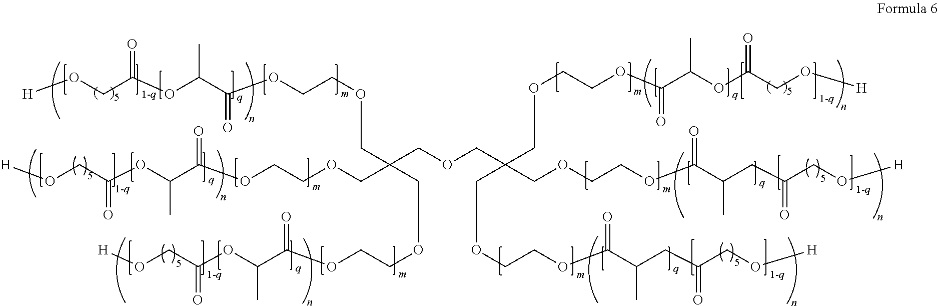
wherein
m is an integer between 5 and 40,
n is an integer between 10 and 115, and
q is between 0.25 and 0.99; 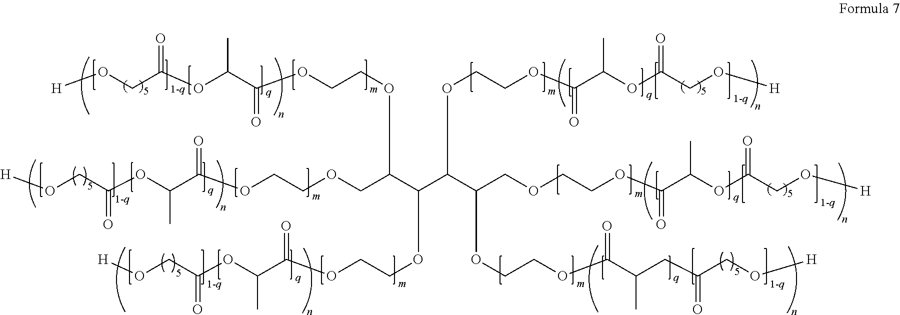
wherein
m is an integer between 5 and 40,
n is an integer between 10 and 115, and
q is between 0.25 and 0.99; 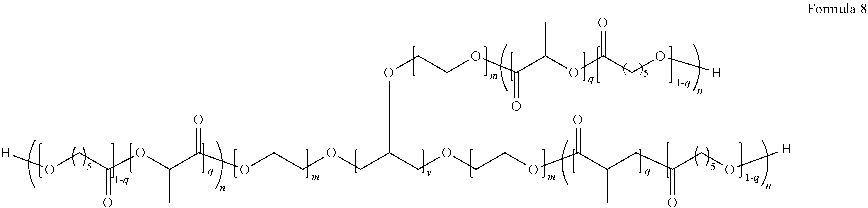
wherein
m is an integer between 25 and 30,
n is an integer between 10 and 90,
q is between 0.25 and 0.99, and
v is 6.

24. The composition according to claim 23, wherein the multi-branched copolymer has Formula 5, x is 1, and R₃ is alkyl.

25. The composition according to claim 23, wherein the multi-branched copolymer has Formula 5, x is 1, and R₃ is

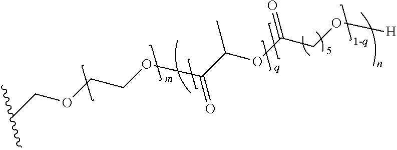

26. The composition according to claim 23, wherein the multi-branched copolymer has Formula 5, x is 0, and R₃ is H.

27. The composition according to claim 23, wherein the multi-branched copolymer has Formula 5, the polyether core has a molecular weight of 2 kDa, and the ester repeat unit to ethylene oxide molar ratio is 4 or 6.

28. The composition according to claim 3, wherein the at least one pharmaceutically active ingredient is in the form of a suspension at a temperature between 10° C. and 37° C.

29. The composition according to claim 3, wherein the molecular weight of the polyether ranges from 0.5 kDa to 10 kDa.

30. The composition according to claim 3, wherein the molar ratio of the ester repeat unit to ethylene oxide of the multi-branched copolymer is from 1 to 10.

31. The composition according to claim 3, wherein the pharmaceutically acceptable organic solvent is a biocompatible organic solvent.

32. The composition according to claim 31, wherein the pharmaceutically acceptable organic solvent is selected from the group consisting of: benzyl alcohol, benzyl benzoate, dimethyl isosorbide (DMI), dimethyl sulfoxide (DMSO), ethyl acetate, ethyl benzoate, ethyl lactate, glycerol formal, methyl ethyl ketone, methyl isobutyl ketone, N-ethyl-2-pyrrolidone, N-methyl-2-pyrrolidinone (NMP), pyrrolidone-2, tetraglycol, triacetin, tributyrin, tripropionin, glycofurol, and mixtures thereof.

33. The composition according to claim 3, wherein the pharmaceutically active ingredient is hydrophobic.

34. The composition according to claim 3, wherein the pharmaceutically active ingredient is meloxicam, tamsulosin, or combinations thereof.

35. The composition according to claim 3, wherein the at least one pharmaceutically active ingredient is present in an amount of from 0.05% to 60% (w/w %) of the total composition.

36. The composition according to claim 3, wherein the composition is an injectable liquid.

37. The composition according to claim 3, wherein the multi-branched copolymer is present in an amount of 20% to 70% (w/w %) of the total composition.

38. (canceled)

39. (canceled)

40. A method of producing the pharmaceutical composition of claim 3, said method comprising dissolving the multi-branched copolymer in a pharmaceutically acceptable organic solvent, and subsequently adding a pharmaceutically active ingredient to the composition.

41. (canceled)

42. (canceled)
